(19) United States
(12) Patent Application Publication
(10) Pub. No.: US 2024/0052351 A1
(43) Pub. Date: Feb. 15, 2024

(54) ANTISENSE OLIGONUCLEOTIDES AND THEIR USE FOR THE TREATMENT OF PAIN

(71) Applicants: INSERM (INSTITUT NATIONAL DE LA SANTÉ DE LA RECHERCHE MÉDICALE), Paris (FR); CENTRE NATIONAL DE LA RECHERCHE SCIENTIFIQUE (CNRS), Paris (FR); UNIVERSITÉ DE BORDEAUX, Bordeaux (FR); UNIVERSITÉ DE MONTPELLIER, Montpellier (FR)

(72) Inventors: Stéphanie Venteo, Montpellier (FR); Patrick Carroll, Montpellier (FR); Philippe BARTHELEMY, Bordeaux (FR); Sébastien BENIZRI, Aixen-Provence Cedex 04 (FR); Alexandre Pattyn, Montpellier (FR)

(21) Appl. No.: 17/766,235

(22) PCT Filed: Oct. 9, 2020
(86) PCT No.: PCT/EP2020/078395

§ 371 Date: Apr. 2, 2022

(30) Foreign Application Priority Data
Oct. 10, 2019 (EP) 19306327.8

(51) Int. Cl. C12N 15/113; A61P 25/04

(52) U.S. Cl. CPC C12N 15/1137; A61P 25/04; C12N 2310/14; C12N 2310/531; C12N 2310/141; C12N 2310/12; C12N 2310/11; C12N 2310/321; C12N 2310/315

(57) ABSTRACT

The invention relates to an inhibitor of FXYD2 wherein said inhibitor reduces the expression and/or activity of FXYD2 in a subject in need thereof and targets at least the region comprising or consisting of the nucleotides 219-229 of SEQ ID NO: 3. Inventors have shown that targeting a region of FXYD2 can be used to inhibit and/or reduce the expression and/or activity of FXYD2. They have designed and synthesized an antisense oligonucleotide (SEQ ID NO: 4) targeting the rat and human FXYD2 gene. The have performed intrathecal injection of FXYD2 optimized ASO in two rat models of pain (neuropathic and inflammatory pain). They have shown that FXYD2 ASO efficiently reduces its expression in rat Dorsal root ganglion (DRG). The have demonstrated that FXYD2 ASO dramatically reduces neuropathic pain in the Spinal Nerve Ligation (SNL) rat model and analgesic effect of FXYD2 ASO on neuropathic pain is greater than that of Ziconotide, the current market leader. They also have shown that FXYD2 ASO dramatically reduces inflammatory pain in Complete Freund's Adjuvant (CFA)-induced rat model.

### FIELD OF THE INVENTION

The invention is in the field of pain, more particularly the invention relates to methods and pharmaceutical compositions for treating peripheral pain.

### BACKGROUND OF THE INVENTION

Pain is an unpleasant feeling often caused by intense or damaging stimuli. The International Association for the Study of Pain's widely used definition states: “Pain is an unpleasant sensory and emotional experience associated with actual or potential tissue damage, or described in terms of such damage”. There are different types of pain.

Acute pain is short-term pain that comes on suddenly and has a specific cause, usually tissue injury. Generally, it lasts for fewer than six months and goes away once the underlying cause is treated.

Chronic pain is a common problem that constitutes a major challenge to healthcare providers because of its complex natural history, unclear etiology, and poor response to therapy. Chronic pain is a poorly defined condition. Most authors consider ongoing pain lasting longer than 6 months as diagnostic, whereas others have used 3 months as the minimum criterion. In chronic pain, the duration parameter is used arbitrarily. Various neuromuscular, reproductive, gastrointestinal, and urologic disorders may cause or contribute to chronic pain.

Nociceptive pain is the most common type of pain. It's caused by stimulation of nociceptors, which are pain receptors for tissue injury. Nociceptors throughout the body, especially in skin and internal organs. When they're stimulated by potential harm, such as a cut or other injury, they send electrical signals to brain, causing the subject to feel the pain.

Visceral pain results from injuries or damage to internal organs. A subject can feel it in the trunk area of his body, which includes your chest, abdomen, and pelvis. It's often hard to pinpoint the exact location of visceral pain. Visceral pain is often described as: pressure, aching, squeezing or cramping.

Somatic pain results from stimulation of the pain receptors in tissues, rather than internal organs. This includes skin, muscles, joints, connective tissues, and bones. It's often easier to pinpoint the location of somatic pain rather than visceral pain. Somatic pain usually feels like a constant aching or gnawing sensation. It can be further classified as either deep or superficial: Deep somatic pain is felt in your joints, tendons, bones, and muscle. It's often described as aching; Superficial somatic pain is felt in your skin and mucus membranes. It may feel sharp or throbbing.

Peripheral neuropathic pain is caused by damage of neural structures from the peripheral nervous system, such as damage to peripheral nerve endings in the skin (e.g. from nociceptors). These damaged nerve endings can generate impulses in the absence of stimulation, can be hypersensitive to normal stimulation, and/or can be triggered by remaining local inflammatory stimulation. Even a very small number of damaged and overactive small nerve fibers in the epidermis are sufficient to trigger peripheral neuropathic pain. Examples are peripheral neuropathic pain due to diabetic neuropathy, post-herpetic neuralgia, trigeminal neuralgia, chronic idiopathic axonal polyneuropathy and chemotherapy induced polyneuropathy.

Two most commonly used topical compounds in the treatment of neuropathic pain are capsaicin (a vanilloid receptor agonist and counter-irritant) and lidocaine (a membrane stabilizer). However, both topical capsaicin 0.025% to 0.075% as well as capsaicin 8% patch, have the disadvantage that application quite often induces intolerable side effects, such as increasing burning sensation, and often the treatment has to be combined with a local anesthetic to neutralize this side effect (Jay GW & Barkin RL (2014)). The topical lidocaine 5% patch, disclosed in U.S. Patent Application 2014/0141056 and in U.S. Patent Application 2013/0184351, needs to be replaced every 12 hours, cannot be used on wounds, ulcers, damaged or inflamed skin, commonly seen in patients with diabetic neuropathy, and might give problems in use when applied to toes, especially in elderly, because the patch has to be cut. Also other topical forms of lidocaine up to 8% in creams and gels are available on the market (Deny S et al. (2014)). Yet no evidence from good quality randomized controlled trials is available to support the use of topical lidocaine to treat neuropathic pain, although some individual studies seem to indicate that topical lidocaine might be effective for relief of neuropathic pain (Derry S et al. (2014)). However, the consensus amongst patients and their medical practitioners is that response rates of patients suffering from neuropathic pain to topical lidocaine and more generally to any neuropathic pain medication, both topically as well as orally, have remained quite unsatisfactory.

More and more it is felt that “neuropathic pain” is an insufficient container concept. “Neuropathic pain” is a collection of different pathological states which are characterized by various pathogenic processes. To expect that one therapeutic molecule will be effective in a series of different neuropathic pain syndromes is clearly a bridge too far. There is thus an urgent need for individualized treatment strategies for patients suffering from specific neuropathic pain conditions. Furthermore, there is a strong need for treatment options with diminished side effects, or even better, without side effects, also when administered chronically, for example a few times per day or week or month, for a period of days, weeks, months, years.

### SUMMARY OF THE INVENTION

The present invention relates to an inhibitor of FXYD2 wherein said inhibitor reduces the expression and/or activity of FXYD2 in a subject in need thereof and targets at least the region comprising or consisting of the nucleotides 219-229 of SEQ ID NO: 3. In particular, the invention is defined by claims.

### DETAILED DESCRIPTION OF THE INVENTION

Inventors have shown that targeting a region of FXYD2 can be used to inhibit and/or reduce the expression and/or activity of FXYD2. They have designed and synthesized an antisense oligonucleotide (ASO; e.g. SEQ ID NO: 17) targeting the rat and human FXYD2 gene. The have performed intrathecal injection of FXYD2 optimized ASO in two rat models of pain (neuropathic and inflammatory pains). They have shown that FXYD2 ASO efficiently reduces its expression in rat Dorsal root ganglion (DRG). The have demonstrated that FXYD2 ASO dramatically reduces neuropathic pain in the Spinal Nerve Ligation (SNL) rat model and that the analgesic effect of FXYD2 ASO on neuropathic pain is greater than that of Ziconotide, the current market leader. They also have shown that FXYD2 ASO dramatically reduces inflammatory pain in the Complete Freund's Adjuvant (CFA)-induced rat model.

Accordingly, the inventors have obtained therapeutic tools to treat pain, more particularly neuropathic pain.

### Sequences of the Invention

In a first aspect, the invention relates to an inhibitor of FXYD2 wherein said inhibitor reduces the expression and/or activity of FXYD2 in a subject in need thereof and targets at least the region comprising or consisting of the nucleotides 219-229 of SEQ ID NO: 3.

As used herein, the term “FXYD2” refers to FXYD domain containing ion transport regulator 2. It has general meaning in the art and refers to the gamma-subunit of the Na,K-ATPase. The term includes naturally occurring FXYD2 variants and modified forms thereof. FXYD2 mRNA sequences can be found in NCBI Gene ID NO: 486.

The naturally occurring human FXYD2 gene, variant b has a nucleotide sequence as shown in Genbank Accession number NM 021603.4. The nucleotide sequence cDNA of Homo sapiens FXYD2, transcript variant b, is defined by the sequence SEQ ID NO: 1 (593 bp):

ACTCTCCATCCAGGCCCCAGGCAAGCAGCACCTCCCTGCTCTCCTGCAC
TCCTGGACACAACCAGCAGCTCCTGCCATGGACAGGTGGTACCTGGGCG
GCAGCCCCAAGGGGGACGTGGACCCGTTCTACTATGACTATGAGACCGT
TCGCAATGGGGGCCTGATCTTCGCTGGACTGGCCTTCATCGTGGGGCTC
CTCATCCTCCTCAGCAGAAGATTCCGCTGTGGGGGCAATAAGAAGCGCA
GGCAAATCAATGAAGATGAGCCGTAACAGCAGCCTCGGCGGTGCCACCC
ACTGCACTGGGGCCAGCTGGGAAGCCAAGCATGGCCCTGCCTCTGGCGC
CTCCCCTTCTTCCCTGGGCTTTAGACCTTTGTCCCCGTCACTGCCAGCG
CTTGGGCTGAAGGAAGCTCCAGACTCAATGTGACCCCCAGGTGGCATCG
CCAACTCCTGCCTCGTGCCACCTCATGCTTATAATAAAGCCGGCGTCAG
AGACCGCTGCTTCCCTCACCTGCCTGCCTGTCTCCCTCCTCTGTCACCA
CCAGCCTCTCCAAGCTCAAGTACAAATACAGCCGGGTCTCATTTGTTTT
TTCAA.

The naturally occurring human FXYD2 gene, variant a, has a nucleotide sequence as shown in Genbank Accession number NM 001680.5. The nucleotide sequence cDNA of Homo sapiens FXYD2, transcript variant a, is defined by the sequence SEQ ID NO: 2 (589 bp):

AGACACTCTCCAAAAAGCAGAGACAGCAGGAAGAGGGGAGTGGAGGCAG
CCCATTCACCTGGGGAAATGACTGGGTTGTCGATGGACGGTGGCGGCAG
CCCCAAGGGGGACGTGGACCCGTTCTACTATGACTATGAGACCGTTCGC
AATGGGGGCCTGATCTTCGCTGGACTGGCCTTCATCGTGGGGCTCCTCA
TCCTCCTCAGCAGAAGATTCCGCTGTGGGGGCAATAAGAAGCGCAGGCA
AATCAATGAAGATGAGCCGTAACAGCAGCCTCGGCGGTGCCACCCACTG
CACTGGGGCCAGCTGGGAAGCCAAGCATGGCCCTGCCTCTGGCGCCTCC
CCTTCTTCCCTGGGCTTTAGACCTTTGTCCCCGTCACTGCCAGCGCTTG
GGCTGAAGGAAGCTCCAGACTCAATGTGACCCCCAGGTGGCATCGCCAA
CTCCTGCCTCGTGCCACCTCATGCTTATAATAAAGCCGGCGTCAGAGAC
CGCTGCTTCCCTCACCTGCCTGCCTGTCTCCCTCCTCTGTCACCACCAG
CCTCTCCAAGCTCAAGTACAAATACAGCCGGGTCTCATTTGTTTTTTCA
A.

The naturally occurring human FXYD2 gene has a common sequence coding for both variants (a and b) has the following nucleotide sequence and defined by the sequence SEQ ID NO: 3:

5′-91
GGCGGCAGCCCCAAGGGGGACGTGGACCCGTTCTACTATGACTATGAGA
CCGTTCGCAATGGGGGCCTGATCTTCGCTGGACTGGCCTTCATCGTGGG
GCTCCTCATCCTCCTCAGCAGAAGATTCCGCTGTGGGGGCAATAAGAAG
CGCAGGCAAATCAATGAAGATGAGCCGTAA 267-3′.

In a particular embodiment, the nucleotide sequence ARN of Homo sapiens FXYD2, transcript variant b, is defined by the sequence SEQ ID NO: 4:

UGA GAG GUA GGU CCG GGG UCC GUU CGU CGU GGA GGG
ACG AGA GGA CGU GAG GAC CUG UGU UGG UCG UCG AGG
ACG GUA CCU GUC CAC CAU GGA CCC GCC GUC GGG GUU
CCC CCU GCA CCU GGG CAA GAU GAU ACU GAU ACU CUG
GCA AGC GUU ACC CCC GGA CUA GAA GCG ACC UGA CCG
GAA GUA GCA CCC CGA GGA GUA GGA GGA GUC GUC UUC
UAA GGC GAC ACC CCC GUU AUU CUU CGC GUC CGU UUA
GUU ACU UCU ACU CGG CAU UGU CGU CGG AGC CGC CAC
GGU GGG UGA CGU GAC CCC GGU CGA CCC UUC GGU UCG
UAC CGG GAC GGA GAC CGC GGA GGG GAA GAA GGG ACC
CGA AAU CUG GAA ACA GGG GCA GUG ACG GUC GCG AAC
CCG ACU UCC UUC GAG GUC UGA GUU ACA CUG GGG GUC
CAC CGU AGC GGU UGA GGA CGG AGC ACG GUG GAG UAC
GAA UAU UAU UUC GGC CGC AGU CUC UGG CGA CGA AGG
GAG UGG ACG GAC GGA CAG AGG GAG GAG ACA GUG GUG
GUC GGA GAG GUU CGA GUU CAU GUU UAU GUC GGC CCA
GAG UAA ACA AAA AAG UU.

In a particular embodiment, the nucleotide sequence ARN of Homo sapiens FXYD2, transcript variant a, is defined by the sequence SEQ ID NO: 5:

UCU GUG AGA GGU UUU UCG UCU CUG UCG UCC UUC UCC
CCU CAC CUC CGU CGG GUA AGU GGA CCC CUU UAC UGA
CCC AAC AGC UAC CUG CCA CCG CCG UCG GGG UUC CCC
CUG CAC CUG GGC AAG AUG AUA CUG AUA CUC UGG CAA
GCG UUA CCC CCG GAC UAG AAG CGA CCU GAC CGG AAG
UAG CAC CCC GAG GAG UAG GAG GAG UCG UCU UCU AAG
GCG ACA CCC CCG UUA UUC UUC GCG UCC GUU UAG UUA
CUU CUA CUC GGC AUU GUC GUC GGA GCC GCC ACG GUG
GGU GAC GUG ACC CCG GUC GAC CCU UCG GUU CGU ACC
GGG ACG GAG ACC GCG GAG GGG AAG AAG GGA CCC GAA
AUC UGG AAA CAG GGG CAG UGA CGG UCG CGA ACC CGA
CUU CCU UCG AGG UCU GAG UUA CAC UGG GGG UCC ACC
GUA GCG GUU GAG GAC GGA GCA CGG UGG AGU ACG AAU
AUU AUU UCG GCC GCA GUC UCU GGC GAC GAA GGG AGU
GGA CGG ACG GAC AGA GGG AGG AGA CAG UGG UGG UCG
GAG AGG UUC GAG UUC AUG UUU AUG UCG GCC CAG AGU
AAA CAA AAA AGU U.

The naturally occurring human FXYD2 gene has a common sequence coding for both variants (a and b) has the following nucleotide sequence ARN and defined by the sequence SEQ ID NO: 6:

CCG CCG UCG GGG UUC CCC CUG CAC CUG GGC AAG AUG
AUA CUG AUA CUC UGG CAA GCG UUA CCC CCG GAC UAG
AAG CGA CCU GAC CGG AAG UAG CAC CCC GAG GAG UAG
GAG GAG UCG UCU UCU AAG GCG ACA CCC CCG UUA UUC
UUC GCG UCC GUU UAG UUA CUU CUA CUC GGC AUU

In a further embodiment, the invention relates to an inhibitor of FXYD2 wherein said inhibitor reduces the expression and/or activity of FXYD2 in a subject in need thereof and targets at least 15 nucleotides of SEQ ID NO: 1, SEQ ID NO: 2, SEQ ID NO: 3, SEQ ID NO: 4, SEQ ID NO: 5 or SEQ ID NO: 6.

In a particular embodiment, the antisense oligonucleotide according to the invention targets:
the region comprising or consisting of nucleotides 210-238 of SEQ ID NO:3; and/or
the region comprising or consisting of nucleotides 210-267 of SEQ ID NO:3.

In a particular embodiment, the inhibitor according to the invention targets at least the region comprising or consisting of the nucleotides of SEQ ID NO: 3.

In a particular embodiment, the invention relates to an inhibitor of FXYD2 wherein said inhibitor reduces the expression and/or activity of FXYD2 in a subject in need thereof and targets at least the region comprising or consisting of the nucleotides of SEQ ID NO: 1, SEQ ID NO: 2, SEQ ID NO: 3, SEQ ID NO: 4, SEQ ID NO: 5, or SEQ ID NO: 6.

In a particular embodiment, the invention relates to an inhibitor of FXYD2 wherein said inhibitor reduces the expression and/or activity of FXYD2 in a subject in need thereof and targets the region comprising or consisting the nucleic acids 219-229 as set for of: SEQ ID NO: 1, SEQ ID NO: 2, SEQ ID NO: 3, SEQ ID NO: 4, SEQ ID NO: 5, or SEQ ID NO: 6.

In a particular embodiment, the invention relates to an inhibitor of FXYD2 wherein said inhibitor reduces the expression and/or activity of FXYD2 in a subject in need thereof and targets the region comprising or consisting the nucleic acids 210-238 as set for of: SEQ ID NO: 1, SEQ ID NO: 2, SEQ ID NO: 3, SEQ ID NO: 4, SEQ ID NO: 5 or SEQ ID NO: 6.

In a particular embodiment, the invention relates to an inhibitor of FXYD2 wherein said inhibitor reduces the expression and/or activity of FXYD2 in a subject in need thereof and targets the region comprising or consisting the nucleic acids 210-267 as set for of: SEQ ID NO: 1, SEQ ID NO: 2, SEQ ID NO: 3, SEQ ID NO: 4, SEQ ID NO: 5 or SEQ ID NO: 6.

In a particular embodiment, the invention relates to an inhibitor of FXYD2 wherein said inhibitor reduces the expression and/or activity of FXYD2 in a subject in need thereof and targets the region comprising the nucleic acids 91 to 267 as set for of SEQ ID NO: 1, SEQ ID NO: 2, SEQ ID NO: 4, SEQ ID NO: 5, or SEQ ID NO: 6.

In a particular embodiment, the invention relates to an inhibitor of FXYD2 wherein said inhibitor reduces the expression and/or activity of FXYD2 in a subject in need thereof and targets the region consisting the nucleic acids 91 to 267 as set for of SEQ ID NO: 1, SEQ ID NO: 2, SEQ ID NO: 4, SEQ ID NO: 5, or SEQ ID NO: 6.

In a particular embodiment, the invention relates to an inhibitor of FXYD2 wherein said inhibitor reduces the expression and/or activity of FXYD2 in a subject in need thereof and targets the region consisting the nucleic acids as set for: SEQ ID NO: 1, SEQ ID NO: 2, SEQ ID NO: 3, SEQ ID NO: 4, SEQ ID NO: 5, or SEQ ID NO: 6.

In a particular embodiment, the invention relates to an inhibitor of FXYD2 wherein said inhibitor reduces the expression and/or activity of FXYD2 in a subject in need thereof and targets the region comprising of the nucleic acids as set for: SEQ ID NO: 1, SEQ ID NO: 2, SEQ ID NO: 3, SEQ ID NO: 4, SEQ ID NO: 5, or SEQ ID NO: 6.

In a particular embodiment, the antisense oligonucleotide according to the invention targets the nucleotide sequence as defined by SEQ ID NO:1 SEQ ID NO: 2, SEQ ID NO: 3, SEQ ID NO: 4, SEQ ID NO: 5 and/or SEQ ID NO: 6.

In some embodiments, the oligonucleotide of the present invention has a length of at least 15 nucleotides.

In some embodiments, the oligonucleotide of the present invention has a length from 15 to 25 nucleotides.

In particular, the oligonucleotide of the present invention has a length of 15, 16, 17, 18, 19, 20, 21, 22, 23, 24 or 25 nucleotides.

As used herein, the term “inhibitor” refers to a natural or synthetic compound that has a biological effect to inhibit or reduce the expression and/or activity of FXYD2.

In a particular embodiment, the inhibitor of gene expression refers to a natural or synthetic compound that has a biological effect to reduce and/or inhibit the expression of FXYD2 gene. It will be understood to the skilled in the art that inhibiting expression of a gene, e.g. the FXYD2 gene, typically results in a decrease or even abolition of the gene product (protein, e.g. FXYD2 protein) in target cells or tissues, although various levels of inhibition may be achieved. Inhibiting or decreasing expression is typically referred to as knockdown.

In a particular embodiment, the inhibitor of activity of FXYD2 refers to a natural or synthetic compound that has a biological effect to reduce and/or inhibit the activity of FXYD2.

In a particular embodiment, the inhibitor of FXYD2 gene expression is a siRNA, a shRNA, an antisense oligonucleotide, miRNA or a ribozyme.

In one embodiment, the inhibitor of FXYD2 according to the invention is a siRNA.

Small inhibitory RNAs, also referred to as short interfering RNAs (siRNAs) can also function as FXYD2 expression inhibitors for use in the present invention. FXYD2 gene expression can be reduced by treating the subject or cell with a small double stranded RNA (dsRNA), or a vector or construct causing the production of a small double stranded RNA, such that FXYD2 expression is specifically inhibited (i.e. RNA interference or RNAi) by degradation of mRNAs in a sequence specific manner. Methods for selecting an appropriate dsRNA or dsRNA-encoding vector are known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836, each of which is incorporated by reference herein in its entirety).

In a particular embodiment, the invention relates to an inhibitor of FXYD2 wherein said inhibitor is siRNA.

In a particular embodiment, the siRNA according to the invention targets the region comprising or consisting the nucleic acids as set for SEQ ID NO: 1, SEQ ID NO: 2, SEQ DI NO: 3, SEQ DI NO: 4, SEQ ID NO: 5 or SEQ DI NO: 6.

In a particular embodiment, the siRNA according to the invention targets the region comprising or consisting the nucleic acids as set for SEQ ID NO: 3.

In a particular embodiment, the siRNA according to the invention which consists of a sequence consisting of SEQ ID NO: 31. The nucleic acids as set for SEQ ID NO: 31 is defined by the following nucleic acids:

5′ AAGAUUCCGCUGUGGGGGC(UU) 3′.

In one embodiment, the inhibitor of FXYD2 according to the invention is a shRNA.

Short hairpin RNAs (shRNA) can also function as FXYD2 expression inhibitors for use in the present invention. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound.

In one embodiment, the inhibitor of FXYD2 according to the invention is a miRNA.

miRNAs (miR) can also function as FXYD2 expression inhibitors for use in the present invention. miRNA has its general meaning in the art and refers to microRNA molecules that are generally 21 to 22 nucleotides in length, even though lengths of 19 and up to 23 nucleotides have been reported, and suppress translation of targeted mRNAs. miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem. The processed miRNA (also referred to as “mature miRNA”) becomes part of a large complex to downregulate, e.g. decrease translation, of a particular target gene.

Multiple miRNAs may be employed to knockdown FXYD2. The miRNAs may be complementary to different target transcripts or different binding sites of a target transcript. Polycistronic transcripts may also be utilized to enhance the efficiency of target gene knockdown. In some embodiments, multiple genes encoding the same miRNAs or different miRNAs may be regulated together in a single transcript, or as separate transcripts in a single vector cassette. In one embodiment, the vector is a viral vector, including but not limited to recombinant adeno-associated viral (rAAV) vectors, lentiviral vectors, retroviral vectors and retrotransposon-based vector systems.

In one embodiment, the inhibitor of FXYD2 is an antisense nucleic acid.

Inhibitor of FXYD2 expression of the invention is based on antisense oligonucleotide constructs. Anti-sense oligonucleotides, including antisense RNA molecules and antisense DNA molecules, would act to directly block the translation of FXYD2 mRNA by binding thereto and thus preventing protein translation or by increasing mRNA degradation, thus decreasing the level of FXYD2 proteins, and thus activity, in a cell. For example, antisense oligonucleotides of at least about 15 bases and complementary to unique regions of the mRNA transcript sequence encoding FXYD2 can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732, each of which is incorporated by reference herein in its entirety).

The antisense RNA that is complementary to the sense target sequence is encoded by a DNA sequence for the production of any of the foregoing inhibitors (e.g., antisense, siRNAs, shRNAs or miRNAs). The DNA encoding double stranded RNA of interest is incorporated into a gene cassette, e.g. an expression cassette in which transcription of the DNA is controlled by a promoter.

In a particular embodiment, the inhibitor of FXYD2 gene expression is an antisense oligonucleotide.

In a particular embodiment, the inhibitor of FXYD2 gene expression is an isolated, synthetic or recombinant antisense oligonucleotide targeting the FXYD2 mRNA transcript. The oligonucleotide of the invention can be of any suitable type.

In some embodiments, the oligonucleotide is a RNA oligonucleotide. In some embodiments, the oligonucleotide is a DNA oligonucleotide.

In a particular embodiment, the antisense oligonucleotide is selected from the group consisting of but not limited to: SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41.

TABLE 1
Sequences of antisense oligonucleotides tested for their capacity
to diminish FXYD2 protein levels in HEK293 cells

| ASO | Position | 5′-3′ Sequence | % GC | SEQ ID NO: |
|---|---|---|---|---|
| 5 | 112-131 | TCATAGTAGAACGGGTCCAC | 50 | SEQ ID NO: 7 |
| 6 | 113-132 | GTCATAGTAGAACGGGTCCA | 50 | SEQ ID NO: 8 |
| 7 | 114-133 | AGTCATAGTAGAACGGGTCC | 50 | SEQ ID NO: 9 |
| 8 | 115-134 | TAGTCATAGTAGAACGGGTC | 45 | SEQ ID NO: 10 |
| 37 | 154-173 | AGTCCAGCGAAGATCAGGCC | 60 | SEQ ID NO: 11 |
| 38 | 155-174 | CAGTCCAGCGAAGATCAGGC | 60 | SEQ ID NO: 12 |
| 39 | 156-175 | CCAGTCCAGCGAAGATCAGG | 60 | SEQ ID NO: 13 |
| 40 | 157-176 | GCCAGTCCAGCGAAGATCAG | 60 | SEQ ID NO: 14 |
| 41 | 158-177 | GGCCAGTCCAGCGAAGATCA | 60 | SEQ ID NO: 15 |
| 74 | 208-227 | CCCCCACAGCGGAATCTTCT | 60 | SEQ ID NO: 16 |
| 75 | 210-229 | TGCCCCCACAGCGGAATCTT | 60 | SEQ ID NO: 17 |
| 76 | 211-230 | TTGCCCCCACAGCGGAATCT | 60 | SEQ ID NO: 18 |
| 77 | 212-231 | ATTGCCCCCACAGCGGAATC | 60 | SEQ ID NO: 19 |
| 78 | 213-232 | TATTGCCCCCACAGCGGAAT | 55 | SEQ ID NO: 20 |
| 79 | 214-233 | TTATTGCCCCCACAGCGGAA | 55 | SEQ ID NO: 21 |
| 80 | 215-234 | CTTATTGCCCCCACAGCGGA | 60 | SEQ ID NO: 22 |
| 81 | 216-235 | TCTTATTGCCCCCACAGCGG | 60 | SEQ ID NO: 23 |
| 82 | 217-236 | TTCTTATTGCCCCCACAGCG | 55 | SEQ ID NO: 24 |
| 83 | 218-237 | CTTCTTATTGCCCCCACAGC | 55 | SEQ ID NO: 25 |
| 84 | 219-238 | GCTTCTTATTGCCCCCACAG | 55 | SEQ ID NO: 26 |
| 86 | 221-240 | GCGCTTCTTATTGCCCCCAC | 60 | SEQ ID NO: 33 |
| 90 | 225-244 | GCCTGCGCTTCTTATTGCCC | 60 | SEQ ID NO: 34 |
| 96 | 231-250 | TGATTTGCCTGCGCTTCTTA | 45 | SEQ ID NO: 35 |
| 105 | 240-259 | CATCTTCATTGATTTGCCTG | 40 | SEQ ID NO: 36 |
| 109 | 244-263 | GGCTCATCTTCATTGATTTG | 40 | SEQ ID NO: 37 |
| 110 | 245-264 | CGGCTCATCTTCATTGATTT | 40 | SEQ ID NO: 27 |
| 111 | 246-265 | ACGGCTCATCTTCATTGATT | 40 | SEQ ID NO: 28 |
| 112 | 247-266 | TACGGCTCATCTTCATTGAT | 40 | SEQ ID NO: 29 |
| 113 | 248-267 | TTACGGCTCATCTTCATTGA | 40 | SEQ ID NO: 30 |
| 75-15 mer | 210-224 | CCACAGCGGAATCTT | 53 | SEQ ID NO: 38 |
| 75-17 mer | 210-226 | CCCCACAGCGGAATCTT | 59 | SEQ ID NO: 39 |
| 82-15 mer | 221-235 | TCTTATTGCCCCCAC | 53 | SEQ ID NO: 40 |
| 82-17 mer | 219-235 | TCTTATTGCCCCCACAG | 53 | SEQ ID NO: 41 |

As used herein, the term “nucleotide” is defined as a modified or naturally occurring deoxyribonucleotide or ribonucleotide. Nucleotides typically include purines and pyrimidines, which include thymidine (T), cytidine (C), guanosine (G), adenosine (A) and uridine (U).

As used herein, the term “oligonucleotide” refers to an oligomer of the nucleotides defined above. The term “oligonucleotide” refers to a nucleic acid sequence, 3′-5′ or 5′-3′ oriented, which may be single- or double-stranded. The oligonucleotide used in the context of the invention may in particular be DNA or RNA. The term also includes “oligonucleotide analog” which refers to an oligonucleotide having (i) a modified backbone structure, e.g., a backbone other than the standard phosphodiester linkage found in natural oligo- and polynucleotides, and (ii) optionally, modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide {e.g., single-stranded RNA or single-stranded DNA). Particularly, analogs are those having a substantially uncharged, phosphorus containing backbone. A substantially uncharged, phosphorus containing backbone in an oligonucleotide analog is one in which a majority of the subunit linkages, e.g., between 50-100%, typically at least 60% to 100% or 75% or 80% of its linkages, are uncharged, and contain a single phosphorous atom.

The term “oligonucleotide” also refers to an oligonucleotide sequence that is inverted relative to its normal orientation for transcription and so correspond to a RNA or DNA sequence that is complementary to a target gene mRNA molecule expressed within the host cell (e.g., it can hybridize to the target gene mRNA molecule through Watson-Crick base pairing).

An antisense strand can be constructed in a number of different ways, provided that it is capable of interfering with the expression of a target gene. For example, the antisense strand can be constructed by reverse-complementing the coding region (or a portion thereof) of the target gene relative to its normal orientation for transcription to allow the transcription of its complement, (e.g., RNAs encoded by the antisense and sense gene may be complementary). In some embodiments, the oligonucleotide need not have the same intron or exon pattern as the target gene, and noncoding segments of the target gene may be equally effective in achieving antisense suppression of target gene expression as coding segments such as antisense oligonucleotide (ASO). In some embodiments, the oligonucleotide has the same exon pattern as the target gene such as siRNA and antisense oligonucleotide (ASO).

As used herein, the term “target” or “targeting” refers to an oligonucleotide able to specifically bind to a FXYD2 gene or a FXYD2 mRNA encoding a FXYD2 gene product. In particular, it refers to an oligonucleotide able to inhibit said gene or said mRNA by the methods known to the skilled in the art (e.g. antisense, RNA interference).

According to the invention, the antisense oligonucleotide of the present invention targets an mRNA and/or DNA encoding FXYD2 gene product, and is capable of reducing the amount of FXYD2 expression and/or activity in cells.

That is to say, the antisense oligonucleotide comprises a sequence that is at least partially complementary, particularly perfectly complementary, to a region of the sequence of said mRNA, said complementarity being sufficient to yield specific binding under intra-cellular conditions. As immediately apparent to the skilled in the art, by a sequence that is “perfectly complementary to” a second sequence is meant the reverse complement counterpart of the second sequence, either under the form of a DNA molecule or under the form of a RNA molecule. A sequence is “partially complementary to” a second sequence if there are one or more mismatches.

The antisense oligonucleotide of the present invention that targets a cDNA or mRNA encoding FXYD2 gene (e.g. FXYD2 gene comprising SEQ ID NO: 1, SEQ ID NO: 2, SEQ ID NO: 3 SEQ ID NO: 4, SEQ ID NO: 5 or SEQ ID NO: 6) can be designed by using the sequence of said mRNA as a basis, e.g. using bioinformatic tools.

Particularly, the antisense oligonucleotide according to the invention is capable of reducing the expression and/or activity of FXYD2 in DRG. Methods for determining whether an oligonucleotide is capable of reducing the expression and/or activity of FXYD2 in cells are known to those skilled in the art.

This can be performed for example be done by analyzing FXYD2 RNA expression such as by RT-qPCR, in situ hybridization or FXYD2 protein expression such as by immunohistochemistry, Western blot, and by comparing FXYD2 protein expression or FXYD2 functional activity in the presence and in the absence of the antisense oligonucleotide to be tested.

In other embodiments, the oligonucleotide is targeted to a translation initiation site (AUG codon), sequences in the coding region (e.g. one or more exons), 5′-untranslated region or 3′-untranslated region of an mRNA. The aim is to interfere with functions of the messenger RNA include all vital functions including translocation of the RNA to the site for protein translation, actual translation of protein from the RNA, splicing or maturation of the RNA and possibly even independent catalytic activity which may be engaged in by the RNA. The overall effect of such interference with the RNA function is to cause interference with protein expression.

In some embodiments, the oligonucleotide of the present invention is further modified, particularly chemically modified, in order to increase the stability and/or therapeutic efficiency in vivo. The one skilled in the art can easily provide some modifications that will improve the efficacy of the oligonucleotide such as stabilizing modifications (C. Frank Bennett and Eric E.

Swayze, RNA Targeting Therapeutics: Molecular Mechanisms of Antisense Oligonucleotides as a Therapeutic Platform Annu. Rev. Pharmacol. Toxicol. 2010.50:259-293; Juliano RL. The delivery of therapeutic oligonucleotides. Nucleic Acids Res. 2016 Aug. 19; 44(14):6518-48). In particular, the oligonucleotide used in the context of the invention may comprise modified nucleotides. Chemical modifications may occur at three different sites: (i) at phosphate groups, (ii) on the sugar moiety, and/or (iii) on the entire backbone structure of the oligonucleotide. Typically, chemical modifications include backbone modifications, heterocycle modifications, sugar modifications, and conjugation strategies.

For example the oligonucleotide is be selected from the group consisting of oligodeoxyribonucleotides, oligoribonucleotides, small regulatory RNAs (sRNAs), U7- or U1-mediated ASOs or conjugate products thereof such as peptide-conjugated or nanoparticle-complexed ASOs, chemically modified oligonucleotide by backbone modifications such as morpholinos, phosphorodiamidate morpholino oligomers (Phosphorodiamidate morpholinos, PMO), peptide nucleic acid (PNA), phosphorothioate (PS) oligonucleotides, stereochemically pure phosphorothioate (PS) oligonucleotides, phosphoramidates modified oligonucleotides, thiophosphoramidate-modified oligonucleotides, and methylphosphonate modified oligonucleotides; chemically modified oligonucleotide by heterocycle modifications such as bicycle modified oligonucleotides, Bicyclic Nucleic Acid (BNA), tricycle modified oligonucleotides, tricyclo-DNA-antisense oligonucleotides (ASOs), nucleobase modifications such as 5-methyl substitution on pyrimidine nucleobases, 5-substituted pyrimidine analogues, 2-Thio-thymine modified oligonucleotides, and purine modified oligonucleotides; chemically modified oligonucleotide by sugar modifications such as Locked Nucleic Acid (LNA) oligonucleotides, 2′,4′-Methyleneoxy Bridged Nucleic Acid (BNA), ethylene-bridged nucleic acid (ENA), constrained ethyl (cEt) oligonucleotides, 2′-Modified RNA, 2′- and 4′-modified oligonucleotides such as 2′-O-Me RNA (2′-OMe), 2′-O-Methoxyethyl RNA (MOE), 2′-Fluoro RNA (FRNA), and 4′-Thio-Modified DNA and RNA; chemically modified oligonucleotide by conjugation strategies such as N-acetyl galactosamine (GalNAc) oligonucleotide conjugates such as 5′-GalNAc and 3′-GalNAc ASO conjugates, lipid oligonucleotide conjugates (LASO), cell penetrating peptides (CPP) oligonucleotide conjugates, targeted oligonucleotide conjugates, antibody-oligonucleotide conjugates, polymer-oligonucleotide conjugate such as with PEGylation and targeting ligand; and chemical modifications and conjugation strategies described for example in Bennett and Swayze, 2010 (RNA targeting therapeutics: molecular mechanisms of antisense oligonucleotides as a therapeutic platform. Annu Rev Pharmacol Toxicol. 2010; 50:259-93); Wan and Seth, 2016 (The Medicinal Chemistry of Therapeutic Oligonucleotides. J Med Chem. 2016 Nov. 10; 59(21):9645-9667); Juliano, 2016 (The delivery of therapeutic oligonucleotides. Nucleic Acids Res. 2016 Aug. 19; 44(14):6518-48); Lundin et al., 2015 (Oligonucleotide Therapies: The Past and the Present. Hum Gene Ther. 2015 August; 26(8):475-85); and Prakash, 2011 (An overview of sugar-modified oligonucleotides for antisense therapeutics. Chem Biodivers. 2011 September; 8(9):1616-41). Indeed, for use in vivo, the oligonucleotide may be stabilized. A “stabilized” oligonucleotide refers to an oligonucleotide that is relatively resistant to in vivo degradation (e.g. via an exo- or endo-nuclease). Stabilization can be a function of length or secondary structure. In particular, oligonucleotide stabilization can be accomplished via phosphate backbone modifications, phosphodiester modifications, phosphorothioate (PS) backbone modifications, combinations of phosphodiester and phosphorothioate modifications, thiophosphoramidate modifications, 2′ modifications (2′-O-Me, 2′-O-(2-methoxyethyl) (MOE) modifications and 2′-fluoro modifications), methylphosphonate, methylphosphorothioate, phosphorodithioate, p-ethoxy, and combinations thereof.

In a particular embodiment, the antisense oligonucleotide is lipid-conjugated, known as LASO. In some embodiments, the antisense oligonucleotide of the present invention is modified by substitution at the 3′ or the 5′ end by a moiety comprising at least three saturated or unsaturated, particularly saturated, linear or branched, particularly linear, hydrocarbon chains comprising from 2 to 30 carbon atoms, particularly from 5 to 20 carbon atoms, more particularly from 10 to 18 carbon atoms as described in WO2014/195432.

In some embodiments, the antisense oligonucleotide of the present invention is modified by substitution at the 3′ or the 5′ end by a moiety comprising at least one ketal functional group, wherein the ketal carbon of said ketal functional group bears two saturated or unsaturated, particularly saturated, linear or branched, particularly linear, hydrocarbon chains comprising from 1 to 22 carbon atoms, particularly from 6 to 20 carbon atoms, in particular 10 to 19 carbon atoms, and even more particularly from 12 to 18 carbon atoms as described in WO2014/195430.

For example, the oligonucleotide may be employed as phosphorothioate derivatives (replacement of a non-bridging phosphoryl oxygen atom with a sulfur atom), which have increased resistance to nuclease digestion. 2′-methoxyethyl (MOE) modification (such as the modified backbone commercialized by IONIS Pharmaceuticals) is also effective. Additionally or alternatively, the oligonucleotide of the present invention may comprise completely, partially or in combination, modified nucleotides which are derivatives with substitutions at the 2′ position of the sugar, in particular with the following chemical modifications: O-methyl group (2′-O-Me) substitution, 2-methoxyethyl group (2′-O-MOE) substitution, fluoro group (2′-fluoro) substitution, chloro group (2′-Cl) substitution, bromo group (2′-Br) substitution, cyanide group (2′-CN) substitution, trifluoromethyl group (2′-CF3) substitution, OCF3 group (2′-OCF3) substitution, OCN group (2′-OCN) substitution, O-alkyl group (2′-O-alkyl) substitution, S-alkyl group (2′-S-alkyl) substitution, N-alkyl group (2′-N-alkyl) substitution, O-alkenyl group (2′-O-alkenyl) substitution, S-alkenyl group (2′-S-alkenyl) substitution, N-alkenyl group (2′-N-alkenyl) substitution, SOCH3 group (2′-SOCH3) substitution, SO2CH3 group (2′-SO2CH3) substitution, ONO2 group (2′-ONO2) substitution, NO2 group (2′-NO2) substitution, N3 group (2′-N3) substitution and/or N12 group (2′-NH2) substitution. Additionally or alternatively, the oligonucleotide of the present invention may comprise completely or partially modified nucleotides wherein the ribose moiety is used to produce locked nucleic acid (LNA), in which a covalent bridge is formed between the 2′ oxygen and the 4′ carbon of the ribose, fixing it in the 3′-endo configuration. These molecules are extremely stable in biological medium, able to activate RNase H such as when LNA are located to extremities (Gapmer) and form tight hybrids with complementary RNA and DNA.

In some embodiments, the oligonucleotide used in the context of the invention comprises modified nucleotides selected from the group consisting of LNA, 2′-OMe analogs, 2′-O-Met, 2′-O-(2-methoxyethyl) (MOE) oligomers, 2′-phosphorothioate analogs, 2′-fluoro analogs, 2′-Cl analogs, 2′-Br analogs, 2′-CN analogs, 2′-CF3 analogs, 2′-OCF3 analogs, 2′-OCN analogs, 2′-O-alkyl analogs, 2′-S-alkyl analogs, 2′-N-alkyl analogs, 2′-O-alkenyl analogs, 2′-S-alkenyl analogs, 2′-N-alkenyl analogs, 2′-SOCH3 analogs, 2′-SO2CH3 analogs, 2′-ONO2 analogs, 2′-NO2 analogs, 2′-N3 analogs, 2′-NH2 analogs, tricyclo (tc)-DNAs, U7 short nuclear (sn) RNAs, tricyclo-DNA-oligoantisense molecules and combinations thereof (U.S. Provisional Patent Application Ser. No. 61/212,384 For: Tricyclo-DNA Antisense Oligonucleotides, Compositions and Methods for the Treatment of Disease, filed Apr. 10, 2009, the complete contents of which is hereby incorporated by reference).

In a particular embodiment, the oligonucleotide according to the invention is a LNA oligonucleotide. As used herein, the term “LNA” (Locked Nucleic Acid) (or “LNA oligonucleotide”) refers to an oligonucleotide containing one or more bicyclic, tricyclic or polycyclic nucleoside analogues also referred to as LNA nucleotides and LNA analogue nucleotides. LNA oligonucleotides, LNA nucleotides and LNA analogue nucleotides are generally described in International Publication No. WO 99/14226 and subsequent applications; International Publication Nos. WO 00/56746, WO 00/56748, WO 00/66604, WO 01/25248, WO 02/28875, WO 02/094250, WO 03/006475; U.S. Pat. Nos. 6,043,060, 6,268,490, 6,770,748, 6,639,051, and U.S. Publication Nos. 2002/0125241, 2003/0105309, 2003/0125241, 2002/0147332, 2004/0244840 and 2005/0203042, all of which are incorporated herein by reference. LNA oligonucleotides and LNA analogue oligonucleotides are commercially available from, for example, Proligo LLC, 6200 Lookout Road, Boulder, CO 80301 USA.

Other forms of oligonucleotides of the present invention are oligonucleotide sequences coupled to small nuclear RNA molecules such as U1 or U7 in combination with a viral transfer method based on, but not limited to, lentivirus or adeno-associated virus (Denti, M A, et al, 2008; Goyenvalle, A, et al, 2004).

Other forms of oligonucleotides of the present invention are peptide nucleic acids (PNA). In peptide nucleic acids, the deoxyribose backbone of oligonucleotides is replaced with a backbone more akin to a peptide than a sugar. Each subunit, or monomer, has a naturally occurring or non-naturally occurring base attached to this backbone. One such backbone is constructed of repeating units of N-(2-aminoethyl)glycine linked through amide bonds. Because of the radical deviation from the deoxyribose backbone, these compounds were named peptide nucleic acids (PNAs) (Dueholm et al., New J. Chem., 1997, 21, 19-31). PNA binds both DNA and RNA to form PNA/DNA or PNA/RNA duplexes. The resulting PNA/DNA or PNA/RNA duplexes are bound with greater affinity than corresponding DNA/DNA, DNA/RNA or RNA/RNA duplexes as determined by Tm's. This high thermal stability might be attributed to the lack of charge repulsion due to the neutral backbone in PNA. The neutral backbone of the PNA also results in the Tm's of PNA/DNA(RNA) duplex being practically independent of the salt concentration. Thus the PNA/DNA(RNA) duplex interaction offers a further advantage over DNA/DNA, DNA/RNA or RNA/RNA duplex interactions which are highly dependent on ionic strength. Homopyrimidine PNAs have been shown to bind complementary DNA or RNA in an anti-parallel orientation forming (PNA)2/DNA(RNA) triplexes of high thermal stability (see, e.g., Egholm, et al., Science, 1991, 254, 1497; Egholm, et al., J. Am. Chem. Soc., 1992, 114, 1895; Egholm, et al., J. Am. Chem. Soc., 1992, 114, 9677). In addition to increased affinity, PNA has also been shown to bind to DNA or RNA with increased specificity. When a PNA/DNA duplex mismatch is melted relative to the DNA/DNA duplex there is seen an 8 to 20° C. drop in the Tm. This magnitude of a drop in Tm is not seen with the corresponding DNA/DNA duplex with a mismatch present. The binding of a PNA strand to a DNA or RNA strand can occur in one of two orientations. The orientation is said to be anti-parallel when the DNA or RNA strand in a 5′ to 3′ orientation binds to the complementary PNA strand such that the carboxyl end of the PNA is directed towards the 5′ end of the DNA or RNA and amino end of the PNA is directed towards the 3′ end of the DNA or RNA. In the parallel orientation the carboxyl end and amino end of the PNA are just the reverse with respect to the 5′-3′ direction of the DNA or RNA. A further advantage of PNA compared to oligonucleotides is that their polyamide backbones (having appropriate nucleobases or other side chain groups attached thereto) is not recognized by either nucleases or proteases and are not cleaved. As a result, PNAs are resistant to degradation by enzymes unlike nucleic acids and peptides. WO92/20702 describes a peptide nucleic acid (PNA) compounds which bind complementary DNA and RNA more tightly than the corresponding DNA. PNA have shown strong binding affinity and specificity to complementary DNA (Egholm, M., et al., Chem. Soc., Chem. Commun., 1993, 800; Egholm, M., et. al., Nature, 1993, 365, 566; and Nielsen, P., et. al. Nucl. Acids Res., 1993, 21, 197). Furthermore, PNA's show nuclease resistance and stability in cell-extracts (Demidov, V. V., et al., Biochem. Pharmacol., 1994, 48, 1309-1313). Modifications of PNA include extended backbones (Hyrup, B., et. al. Chem. Soc., Chem. Commun., 1993, 518), extended linkers between the backbone and the nucleobase, reversal of the amida bond (Lagriffoul, P. H., et. al., Biomed. Chem. Lett., 1994, 4, 1081), and the use of a chiral backbone based on alanine (Dueholm, K. L, et. al., Bio Med. Chem. Lett., 1994, 4, 1077). Peptide Nucleic Acids are described in U.S. Pat. Nos. 5,539,082 and 5,539,083. Peptide Nucleic Acids are further described in U.S. patent application Ser. No. 08/686,113.

Typically, the oligonucleotides of the present invention are obtained by conventional methods well known to those skilled in the art. For example, the oligonucleotide of the invention can be synthesized de novo using any of a number of procedures well known in the art. For example, the b-cyanoethyl phosphoramidite method (Beaucage et al., 1981); nucleoside H-phosphonate method (Garegg et al., 1986; Froehler et al., 1986, Garegg et al., 1986, Gaffney et al., 1988). These chemistries can be performed by a variety of automated nucleic acid synthesizers available in the market. These nucleic acids may be referred to as synthetic nucleic acids. Alternatively, oligonucleotide can be produced on a large scale in plasmids (see Sambrook, et al., 1989). Oligonucleotide can be prepared from existing nucleic acid sequences using known techniques, such as those employing restriction enzymes, exonucleases or endonucleases. Oligonucleotide prepared in this manner may be referred to as isolated nucleic acids.

The one skilled in the art can easily provide some approaches and modifications for enhancing the delivery and the efficacy of oligonucleotides such as chemical modification of the oligonucleotides, lipid- and polymer-based nanoparticles or nanocarriers, ligand-oligonucleotide conjugates by linking oligonucleotides to targeting agents such as carbohydrates, peptides, antibodies, aptamers, lipids or small molecules and small molecules that improve oligonucleotide delivery such as described in Juliano RL. The delivery of therapeutic oligonucleotides. Nucleic Acids Res. 2016 Aug. 19; 44(14):6518-48. Lipophilic conjugates and lipid conjugates include fatty acid-oligonucleotide conjugates; sterol-oligonucleotide conjugates and vitamin-oligonucleotide conjugates.

In a particular embodiment, the oligonucleotide of the present invention is conjugated to a second molecule. Typically said second molecule is selected from the group consisting of aptamers, antibodies or polypeptides. For example, the oligonucleotide of the present invention may be conjugated to a cell penetrating peptide. Cell penetrating peptides are well known in the art and include for example the TAT peptide (Bechara C, Sagan S. Cell-penetrating peptides: 20 years later, where do we stand? FEBS Lett. 2013 Jun. 19; 587(12):1693-702).

In some embodiments, the oligonucleotide of the present invention is associated with a carrier or vehicle, e.g., liposomes or micelles, although other carriers could be used, as would be appreciated by one skilled in the art. Liposomes are vesicles made of a lipid bilayer having a structure similar to biological membranes. Such carriers are used to facilitate the cellular uptake or targeting of the oligonucleotide, or improve the oligonucleotide's pharmacokinetic or therapeutic properties. For example, the oligonucleotide of the present invention may also be administered encapsulated in liposomes, pharmaceutical compositions wherein the active ingredient is contained either dispersed or variously present in corpuscles consisting of aqueous concentric layers adherent to lipidic layers. The oligonucleotide, depending upon solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such as diacetylphosphate, stearylamine, or phosphatidic acid, or other materials of a hydrophobic nature. The diameters of the liposomes generally range from about 15 nm to about 5 microns. The use of liposomes as drug delivery vehicles offers several advantages. Liposomes increase intracellular stability, increase uptake efficiency and improve biological activity. Liposomes are hollow spherical vesicles composed of lipids arranged in a similar fashion as those lipids, which make up the cell membrane. They have an internal aqueous space for entrapping water soluble compounds and range in size from 0.05 to several microns in diameter. Several studies have shown that liposomes can deliver nucleic acids to cells and that the nucleic acids remain biologically active. For example, a liposome delivery vehicle originally designed as a research tool, such as Lipofectin, can deliver intact nucleic acid molecules to cells. Specific advantages of using liposomes include the following: they are non-toxic and biodegradable in composition; they display long circulation half-lives; and recognition molecules can be readily attached to their surface for targeting to tissues. Finally, cost-effective manufacture of liposome-based pharmaceuticals, either in a liquid suspension or lyophilized product, has demonstrated the viability of this technology as an acceptable drug delivery system.

In some embodiments, the oligonucleotide of the present invention is complexed with a complexing agent to increase cellular uptake of oligonucleotides. An example of a complexing agent includes cationic lipids. Cationic lipids can be used to deliver oligonucleotides to cells. The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In general cationic lipids include saturated and unsaturated alkyl and alicyclic ethers and esters of amines, amides, or derivatives thereof. Straight-chain and branched alkyl and alkenyl groups of cationic lipids can contain, e.g., from 1 to about 25 carbon atoms. Particularly, straight chain or branched alkyl or alkene groups have six or more carbon atoms. Alicyclic groups include cholesterol and other steroid groups. Cationic lipids can be prepared with a variety of counterions (anions) including, e.g., Cl—, Br—, I—, F—, acetate, trifluoroacetate, sulfate, nitrite, and nitrate. Examples of cationic lipids include: polyethylenimine, polyamidoamine (PAMAM) starburst dendrimers, Lipofectin (a combination of DOTMA and DOPE), Lipofectase, Lipofectamine, DOPE, Cytofectin (Gilead Sciences, Foster City, Calif.), and Eufectins (JBL, San Luis Obispo, Calif.). Cationic liposomes may comprise the following: N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3p-[N—(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethy-1-hydroxyethyl ammonium bromide; and dimethyldioctadecylammonium bromide (DDAB). The cationic lipid N-(1-(2,3-dioleyloxy)propyl)-N,N,N-trimethylammonium chloride (DOTMA), for example, was found to increase 1000-fold the antisense effect of a phosphorothioate oligonucleotide. (Vlassov et al., 1994, Biochimica et Biophysica Acta 1197:95-108). Oligonucleotides can also be complexed with, e.g., poly(L-lysine) or avidin and lipids may, or may not, be included in this mixture (e.g., steryl-poly(L-lysine). Cationic lipids have been used in the art to deliver oligonucleotides to cells (see, e.g., U.S. Pat. Nos. 5,855,910; 5,851,548; 5,830,430; 5,780,053; 5,767,099; Lewis et al. 1996. Proc. Natl. Acad. Sci. USA 93:3176; Hope et al. 1998. Molecular Membrane Biology 15:1). Other lipid compositions which can be used to facilitate uptake of the instant oligonucleotides can be used in connection with the claimed methods. In addition to those listed supra, other lipid compositions are also known in the art and include, e.g., those taught in U.S. Pat. Nos. 4,235,871; 4,501,728; 4,837,028; 4,737,323.

In particular embodiment, the inhibitor according to the invention, wherein said inhibitor targets a region consisting of nucleotides of SEQ ID NO: 3.

In a particular embodiment, the antisense oligonucleotide according to the invention which comprises a sequence consisting of: SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40, or SEQ ID NO:41.

In a particular embodiment, the antisense oligonucleotide according to the invention which consists of a sequence consisting of: SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40, or SEQ ID NO:41.

In a particular embodiment, the antisense oligonucleotide according to the invention which comprises and/or consists of a sequence consisting of: SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40, or SEQ ID NO:41.

In a particular embodiment, the antisense oligonucleotide according to the invention which comprises and/or consists of a sequence consisting of SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26.

In a particular embodiment, the inhibitor and/or the antisense oligonucleotide according to the invention, wherein the inhibitor and/or the antisense oligonucleotide is capable of reducing the amount of FYXD2 in Dorsal root ganglion (DRG).

According to the invention a first nucleic acid sequence having at least 70% of identity with a second nucleic acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99% identity to the second nucleic acid sequence. Nucleic acid sequence identity is particularly determined using a suitable sequence alignment algorithm and default parameters, such as BLAST N (Karlin and Altschul, Proc. Natl Acad. Sci. USA 87(6):2264-2268 (1990)).

### Vector of the Invention

In a second aspect, the present invention relates to a vector for delivery of a heterologous nucleic acid, wherein the nucleic acid encodes an inhibitory RNA that specifically binds to FXYD2 mRNA and inhibits expression of FXYD2 in a cell.

In a particular embodiment, the invention relates to a vector for delivery of a heterologous nucleic acid, wherein the nucleic acid encodes for an inhibitor according the invention that specifically binds to FXYD2 mRNA and inhibits expression of FXYD2 in a cell.

In a particular embodiment, the vector according to invention, wherein the inhibitor is a siRNA or an antisense oligonucleotide as described above.

In a further embodiment, the acid nucleic acid (e.g. antisense nucleic acid) of the invention may be delivered in vivo alone (naked ASO/LASO) or in association with a vector.

In its broadest sense, a “vector” is any vehicle capable of facilitating the transfer of the oligonucleotide of the invention to the cells. Particularly, the vector transports the nucleic acid to cells with reduced degradation relative to the extent of degradation that would result in the absence of the vector. In general, the vectors useful in the invention include, but are not limited to, naked plasmids, non-viral delivery systems (cationic transfection agents, liposomes, lipid nanoparticles, and the like), phagemids, viruses, other vehicles derived from viral or bacterial sources that have been manipulated by the insertion or incorporation of the oligonucleotide sequences. Viral vectors include, but are not limited to nucleic acid sequences from the following viruses: RNA viruses such as a retrovirus (as for example moloney murine leukemia virus and lentiviral derived vectors), harvey murine sarcoma virus, murine mammary tumor virus, and rous sarcoma virus; adenovirus, adeno-associated virus (AAV); SV40-type viruses; polyoma viruses; Epstein-Barr viruses; papilloma viruses; herpes virus; vaccinia virus; polio virus. One can readily employ other vectors not named but known to the art.

Accordingly, an object of the invention relates to a vector comprising an oligonucleotide sequence that encodes a portion or fragment of FXYD2, or variants thereof.

In another embodiment, the vector of the invention comprises any variant of the oligonucleotide sequence that encodes a portion or fragment of FXYD2.

In another embodiment, the vector of the invention comprises any variant of the oligonucleotide sequence that encodes any variant of FXYD2.

In another embodiment, the invention relates to a vector comprising an antisense oligonucleotide sequence that encodes a portion or fragment of FXYD2, or variants thereof.

In another embodiment, the invention relates to a vector comprising a shRNA sequence that encodes a portion or fragment of the FXYD2, or variants thereof.

In another embodiment, the invention relates to a vector comprising a miRNA sequence that encodes a portion or fragment of FXYD2, or variants thereof.

In another embodiment, the vector according to the invention, wherein said the antisense oligonucleotide targets the region comprising or consisting of the nucleotides of SEQ ID NO: 1, SEQ ID NO: 2, SEQ ID NO: 3, SEQ ID NO: 4, SEQ ID NO: 5, or SEQ ID NO: 6.

In another embodiment, the invention relates to a vector comprising the sequence SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29 SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41 that encodes a portion or fragment of FXYD2, or variants thereof.

In another embodiment, the invention relates to a vector consisting of: SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30 SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41 that encodes a portion or fragment of FXYD2, or variants thereof.

In another embodiment, the vector of the invention comprises any variant of the sequence: SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29 or SEQ ID NO:30, SEQ ID NO:26 or SEQ ID NO:27 that encodes a portion or fragment of FXYD2, or variants thereof.

In another embodiment, the vector of the invention consists of any variant of the sequence of: SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41 that encodes a portion or fragment of FXYD2, or variants thereof.

In a particular embodiment, the vector of the invention consists of any variant of the sequence of: SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26.

In another embodiment, the invention relates to a vector comprising one sequence selected from the group consisting of but not limited to SEQ ID NO: 1, SEQ ID: 2, SEQ ID NO: 3, SEQ ID NO: 4, SEQ ID NO: 5 or SEQ ID NO: 6 and a promoter.

In another embodiment, the invention relates to a vector comprising or consisting of one sequence selected from the group consisting of but not limited to SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41 and a promoter.

In another embodiment, the invention relates to a vector comprising or consisting of one sequence selected from the group consisting of but not limited to SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26 and a promoter.

In another embodiment, the vector according to the invention, wherein said the antisense oligonucleotide targets the region comprising or consisting of the nucleotides of SEQ ID NO: 1, SEQ ID NO: 2, SEQ ID NO: 3, SEQ ID NO: 4, SEQ ID NO: 5, or SEQ ID NO: 6 and a U6 promoter or a PolII promoter.

In some embodiments, the vector comprises the sequence set forth in comprising one sequence selected from the group consisting of but not limited to SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30 SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41 and a U6 promoter or a PolII promoter.

In some embodiments, the vector comprises the sequence set forth in comprising one sequence selected from the group consisting of but not limited to SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26 and a U6 promoter or a PolII promoter.

In another embodiment, the invention relates to a vector comprising an oligonucleotide sequence that encodes a portion or fragment of FXYD2, or variants thereof and a CAG promoter.

In another embodiment, the invention relates to a vector comprising a miRNA sequence that encodes a portion or fragment of FXYD2, or variants thereof and a CAG promoter or a PolII promoter.

In another embodiment, the invention relates to a vector comprising a shRNA sequence that encodes a portion or fragment of FXYD2, or variants thereof and a U6 promoter.

In another embodiment, the vector according to the invention, wherein said the antisense oligonucleotide targets the region comprising or consisting of the nucleotides of SEQ ID NO: 1, SEQ ID NO: 2, SEQ ID NO: 3, SEQ ID NO: 4, SEQ ID NO: 5, or SEQ ID NO: 6 and a CAG promoter.

In another embodiment, the invention relates to a vector comprising one sequence selected from the group consisting of but not limited to SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41 and a CAG promoter.

In a particular embodiment, the invention a vector comprising one sequence selected from the group consisting of but not limited to SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26 and a CAG promoter.

The variants include, for instance, naturally-occurring variants due to allelic variations between individuals (e.g., polymorphisms), alternative splicing forms, etc. The term variant also includes genes sequences of the invention from other sources or organisms. Variants are preferably substantially homologous to sequences according to the invention, i.e., exhibit a nucleotide sequence identity of typically at least about 75%, preferably at least about 85%, more preferably at least about 90%, more preferably at least about 95% with sequences of the invention. Variants of the genes of the invention also include nucleic acid sequences, which hybridize to a sequence as defined above (or a complementary strand thereof) under stringent hybridization conditions. Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

In a particular embodiment, the vector use according to the invention is a non-viral vector or a viral vector.

In a particular embodiment, the non-viral vector is a plasmid comprising a nucleic acid sequence that encodes FXYD2.

In another particular embodiment, the vector may a viral vector.

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins which mediate cell transduction.

As used herein, the term “transgene” refers to the antisense oligonucleotide of the invention.

The terms “gene transfer” or “gene delivery” refer to methods or systems for reliably inserting foreign DNA into host cells. Such methods can result in transient expression of non-integrated transferred DNA, extrachromosomal replication and expression of transferred replicons (e. g. episomes), or integration of transferred genetic material into the genomic DNA of host cells.

Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478.

In a particular embodiment, the viral vector may be an adenoviral, a retroviral, a lentiviral, a herpesvirus or an adeno-associated virus (AAV) vectors.

In a particular embodiment, adeno-associated viral (AAV) vectors are employed.

In another embodiment, the invention relates to an adeno-associated virus (AAV) vector comprising an oligonucleotide sequence that encodes a portion or fragment FXYD2, or variants thereof.

In another embodiment, the adeno-associated virus (AAV) vector of the invention comprises any variant of the oligonucleotide sequence which encodes a portion or fragment of FXYD2.

In another embodiment, the invention relates to an adeno-associated virus (AAV) vector comprising an antisense sequence that encodes a portion or fragment of FXYD2, or variants thereof.

In a particular embodiment, the invention relates to an adeno-associated virus (AAV) vector comprising a shRNA sequence that encodes a portion or fragment of FXYD2, or variants thereof.

In a particular embodiment, the invention relates to an adeno-associated virus (AAV) vector comprising a miRNA sequence that encodes a portion or fragment of FXYD2, or variants thereof.

In another embodiment, the adeno-associated virus (AAV) according to the invention, wherein said the antisense oligonucleotide targets the region comprising or consisting of the nucleotides of SEQ ID NO: 1, SEQ ID NO: 2, SEQ ID NO: 3, SEQ ID NO: 4, SEQ ID NO: 5, or SEQ ID NO: 6.

In a particular embodiment, the invention relates to an adeno-associated virus (AAV) vector comprising one sequence selected from the group consisting of but not limited to SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41 that encodes a portion or fragment of FXYD2, or variants thereof.

In another embodiment, the adeno-associated virus (AAV) vector of the invention comprises any variant of one sequence selected from the group consisting of but not limited to SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41 which encodes a portion or fragment of FXYD2, or variants thereof.

In a particular embodiment, the adeno-associated virus (AAV) vector of the invention comprises any variant of one sequence selected from the group consisting of but not limited to SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26.

In a particular embodiment, the invention relates to an adeno-associated virus (AAV) vector comprising an oligonucleotide sequence that encodes a portion or fragment of FXYD2, or variants thereof.

In a particular embodiment, the invention relates to an adeno-associated virus (AAV) vector comprising an antisense sequence that encodes a portion or fragment of FXYD2, or variants thereof.

In a particular embodiment, the invention relates to an adeno-associated virus (AAV) vector comprising a miRNA sequence that encodes a portion or fragment of FXYD2, or variants thereof.

In a particular embodiment, the invention relates to an adeno-associated virus (AAV) vector comprising a shRNA sequence that encodes a portion or fragment of FXYD2, or variants thereof.

In a particular embodiment, the invention relates to an adeno-associated virus (AAV) vector comprising an oligonucleotide sequence that encodes a portion or fragment of FXYD2 or variants thereof and a CAG promoter.

In a particular embodiment, the invention relates to an adeno-associated virus (AAV) vector comprising an antisense sequence that encodes a portion or fragment of FXYD2, or variants thereof and a CAG promoter.

In a particular embodiment, the invention relates to an adeno-associated virus (AAV) vector comprising a miRNA sequence that encodes a portion or fragment of FXYD2 or variants thereof and a CAG promoter.

In a particular embodiment, the invention relates to an adeno-associated virus (AAV) vector comprising a shRNA sequence that encodes a portion or fragment of FXYD2 or variants thereof and a CAG promoter.

In a particular embodiment, the invention relates to an adeno-associated virus (AAV) vector comprising an antisense oligonucleotide which targets the region comprising or consisting of the nucleotides of SEQ ID NO: 1, SEQ ID NO: 2, SEQ ID NO: 3, SEQ ID NO: 4, SEQ ID NO: 5, or SEQ ID NO: 6 and a CAG promoter.

In a particular embodiment, the invention relates to an adeno-associated virus (AAV) vector comprising the sequence SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41 and a CAG promoter.

In a particular embodiment, the invention relates to an adeno-associated virus (AAV) vector comprising one sequence selected from the group consisting of but not limited to SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26 and a CAG promoter.

In one embodiment, the AAV vector is AAV1, AAV2, AAV3, AAV4, AAV5, AAV6, AAV7, AAV8, AAV9, AAV10, AAVrh.10 or any other serotypes of AAV that can infect human, rodents, monkeys or other species.

By an “AAV vector” is meant a vector derived from an adeno-associated virus serotype, including without limitation, AAV-1, AAV-2, AAV-3, AAV-4, AAV-5, AAV6, AAV7, AAV8, AAV9, AAV10, AAVrh.10, etc. AAV vectors can have one or more of the AAV wild-type genes deleted in whole or part, e.g. the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e. g., functional ITRs) of the virus. ITRs do not need to be the wild-type polynucleotide sequences, and may be altered, e.g, by the insertion, deletion or substitution of nucleotides, so long as the sequences provide for functional rescue, replication and packaging. AAV expression vectors are constructed using known techniques to at least provide as operatively linked components in the direction of transcription, control elements including a transcriptional initiation region, the DNA of interest (i.e. the nucleic acid sequences of the invention) and a transcriptional termination region.

In certain embodiments the viral vectors utilized in the compositions and methods of the invention are recombinant adeno-associated virus (rAAV). The rAAV may be of any serotype, modification, or derivative, known in the art, or any combination thereof (e.g., a population of rAAV that comprises two or more serotypes, e.g., comprising two or more of rAAV2, rAAV8, and rAAV9) known in the art. In some embodiments, the rAAV are rAAV1, rAAV2, rAAV3, rAAV4, rAAV5, rAAV6, rAAV7, rAAV8, rAAV9, rAAV10, rAAV-11, rAAV-12, rAAV-13, rAAV-14, rAAV-15, rAAV-16, rAAV.rh8, rAAV.rh10, rAAV.rh20, rAAV.rh39, rAAV.Rh74, rAAV.RHM4-1, AAV.hu37, rAAV.Anc80, rAAV.Anc80L65, rAAV.7m8, rAAV.PHP.B, rAAV2.5, rAAV2tYF, rAAV3B, rAAV.LK03, rAAV.HSC1, rAAV.HSC2, rAAV.HSC3, rAAV.HSC4, rAAV.HSC5, rAAV.HSC6, rAAV.HSC7, rAAV.HSC8, rAAV.HSC9, rAAV.HSC10, rAAV.HSC11, rAAV.HSC12, rAAV.HSC13, rAAV.HSC14, rAAV.HSC15, or rAAV.HSC16, or other rAAV, or combinations of two or more thereof.

In some embodiments, the rAAV used in the compositions and methods of the invention comprise a capsid protein from an AAV capsid serotype selected from AAV1, AAV2, AAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. In some embodiments, the rAAV comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., vp1, vp2 and/or vp3 sequence of an AAV capsid serotype selected from AAV1, AAV2, AAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In certain embodiments, the AAV that is used in the methods described herein is Anc80 or Anc80L65, as described in Zinn et al., 2015: 1056-1068, which is incorporated by reference in its entirety. In certain embodiments, the AAV that is used in the methods described herein comprises one of the following amino acid insertions: LGETTRP (SEQ ID NO: 14) or LALGETTRP (SEQ ID NO: 15), as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is AAV.7m8, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is any AAV disclosed in U.S. Pat. No. 9,585,971, such as AAV-PHP.B. In certain embodiments, the AAV that is used in the methods described herein is any AAV disclosed in U.S. Pat. No. 9,840,719 and WO 2015/013313, such as AAV.Rh74 and RHM4-1, each of which is incorporated herein by reference in its entirety. In certain embodiments, the AAV that is used in the methods described herein is any AAV disclosed in WO 2014/172669, such as AAV rh.74, which is incorporated herein by reference in its entirety. In certain embodiments, the AAV that is used in the methods described herein is AAV2/5, as described in Georgiadis et al., 2016, Gene Therapy 23: 857-862 and Georgiadis et al., 2018, Gene Therapy 25: 450, each of which is incorporated by reference in its entirety. In certain embodiments, the AAV that is used in the methods described herein is any AAV disclosed in WO 2017/070491, such as AAV2tYF, which is incorporated herein by reference in its entirety. In certain embodiments, the AAV that is used in the methods described herein is AAVLK03 or AAV3B, as described in Puzzo et al., 2017, Sci. Transl. Med. 29(9): 418, which is incorporated by reference in its entirety. In certain embodiments, the AAV that is used in the methods described herein is any AAV disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In certain embodiments, the AAV that is used in the methods described herein is an AAV disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335. In some embodiments, the rAAV have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the vp1, vp2 and/or vp3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, the rAAV have a capsid protein disclosed in Intl. Appl. Publ. No. WO 2003/052051 (see, e.g., SEQ ID NO: 2), WO 2005/033321 (see, e.g., SEQ ID NOs: 123 and 88), WO 03/042397 (see, e.g., SEQ ID NOs: 2, 81, 85, and 97), WO 2006/068888 (see, e.g., SEQ ID NOs: 1 and 3-6), WO 2006/110689, (see, e.g., SEQ ID NOs: 5-38) WO2009/104964 (see, e.g., SEQ ID NOs: 1-5, 7, 9, 20, 22, 24 and 31), WO 2010/127097 (see, e.g., SEQ ID NOs: 5-38), and WO 2015/191508 (see, e.g., SEQ ID NOs: 80-294), and U.S. Appl. Publ. No. 20150023924 (see, e.g., SEQ ID NOs: 1, 5-10), the contents of each of which is herein incorporated by reference in its entirety. In some embodiments, the rAAV have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the vp1, vp2 and/or vp3 sequence of an AAV capsid disclosed in Intl. Appl. Publ. No. WO 2003/052051 (see, e.g., SEQ ID NO: 2), WO 2005/033321 (see, e.g., SEQ ID NOs: 123 and 88), WO 03/042397 (see, e.g., SEQ ID NOs: 2, 81, 85, and 97), WO 2006/068888 (see, e.g., SEQ ID NOs: 1 and 3-6), WO 2006/110689 (see, e.g., SEQ ID NOs: 5-38) WO2009/104964 (see, e.g., SEQ ID NOs: 1-5, 7, 9, 20, 22, 24 and 31), WO 2010/127097 (see, e.g., SEQ ID NOs: 5-38), and WO 2015/191508 (see, e.g., SEQ ID NOs: 80-294), and U.S. Appl. Publ. No. 20150023924 (see, e.g., SEQ ID NOs: 1, 5-10).

Nucleic acid sequences of AAV based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; International Patent Application Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

In additional embodiments, the rAAV comprise a pseudotyped rAAV. In some embodiments, the pseudotyped rAAV are rAAV2/8 or rAAV2/9 pseudotyped rAAV. Methods for producing and using pseudotyped rAAV are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, the rAAV comprise a capsid containing a capsid protein chimeric of two or more AAV capsid serotypes. In some embodiments, the capsid protein is a chimeric of 2 or more AAV capsid proteins from AAV serotypes selected from AAV1, AAV2, AAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10 AAV.HSC11, AAV.HSC12, AAV.HSC13, AAV.HSC14, AAV.HSC15, or AAV.HSC16.

In certain embodiments, a single-stranded AAV (ssAAV) can be used. In certain embodiments, a self-complementary vector, e.g., scAAV, can be used (see, e.g., Wu, 2007, Human Gene Therapy, 18(2):171-82, McCarty et al, 2001, Gene Therapy, Vol. 8, Number 16, Pages 1248-1254; and U.S. Pat. Nos. 6,596,535; 7,125,717; and 7,456,683, each of which is incorporated herein by reference in its entirety).

In certain embodiments, the recombinant AAV vector used for delivering the transgene have a tropism for cells in the DRG. Such vectors can include non-replicating “rAAV”, particularly those bearing an AAV8 or AAVrh10 capsid are preferred. In certain embodiments, the viral vectors provided herein are AAV9 or AAVrh10 based viral vectors. In certain embodiments, the AAV8 or AAVrh10 based viral vectors provided herein retain tropism for DRG. AAV variant capsids can be used, including but not limited to those described by Wilson in U.S. Pat. No. 7,906,111 which is incorporated by reference herein in its entirety, with AAV/hu.31 and AAV/hu.32 being particularly preferred; as well as AAV variant capsids described by Chatterjee in U.S. Pat. Nos. 8,628,966, 8,927,514 and Smith et al., 2014, Mol Ther 22: 1625-1634, each of which is incorporated by reference herein in its entirety.

In some embodiment, the present invention relates to a recombinant adeno-associated virus (rAAV) comprising (i) an expression cassette containing a transgene under the control of regulatory elements and flanked by ITRs, and (ii) an AAV capsid, wherein the transgene encodes an inhibitory RNA that specifically binds FXYD2 mRNA and inhibits expression of FXYD2 in a cell.

Provided in particular embodiments are AAV vectors comprising an artificial genome comprising (i) an expression cassette containing the transgene under the control of regulatory elements and flanked by ITRs; and (ii) a viral capsid that has the amino acid sequence of the AAV capsid protein or is at least 95%, 96%, 97%, 98%, 99% or 99.9% identical to the amino acid sequence of the AAV capsid protein while retaining the biological function of the AAV capsid.

Provided in particular embodiments are AAVrh10 vectors comprising an artificial genome comprising (i) an expression cassette containing the transgene under the control of regulatory elements and flanked by ITRs; and (ii) a viral capsid that has the amino acid sequence of the AAVrh10 capsid protein or is at least 95%, 96%, 97%, 98%, 99% or 99.9% identical to the amino acid sequence of the AAVrh10 capsid protein while retaining the biological function of the AAVrh10 capsid. In certain embodiments, the encoded AAVrh10 capsid has the sequence of SEQ ID NO: 81 set forth in U.S. Pat. No. 9,790,427 which is incorporated by reference herein in its entirety, with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAVrh10 capsid.

The control elements are selected to be functional in a mammalian cell. The resulting construct which contains the operatively linked components is flanked by (5′ and 3′) functional AAV ITR sequences. By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a polynucleotide sequence interposed between two flanking ITRs into a mammalian cell genome. The polynucleotide sequences of AAV ITR regions are known. As used herein, an “AAV ITR” does not necessarily comprise the wild-type polynucleotide sequence, but may be altered, e. g., by the insertion, deletion or substitution of nucleotides.

Additionally, the AAV ITR may be derived from any of several AAV serotypes, including without limitation, AAV-1, AAV-2, AAV-3, AAV-4, AAV-5, AAV6, AAV7, AAV8, AAV9, AAV10, AAVrh.10, etc. Furthermore, 5′ and 3′ ITRs which flank a selected polynucleotide sequence in an AAV vector need not necessarily be identical or derived from the same AAV serotype or isolate, so long as they function as intended, i. e., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV-1, AAV-2, AAV-3, AAV-4, AAV 5, AAV6, AAV7, AAV8, AAV9, AAV10, AAVrh.10, etc. Furthermore, 5′ and 3′ ITRs which flank a selected polynucleotide sequence in an AAV expression vector need not necessarily be identical or derived from the same AAV serotype or isolate, so long as they function as intended, i. e., to allow for excision and rescue of the sequence of interest from a host cell genome or vector, and to allow integration of the DNA molecule into the recipient cell genome when AAV Rep gene products are present in the cell.

Particular embodiments are vectors derived from AAV serotypes having tropism for and high transduction efficiencies in cells of the mammalian DRG. A review and comparison of transduction efficiencies of different serotypes is provided in this patent application. In certain examples, AAV2, AAV5, AAV8, AAV9 and rh.10 based vectors direct long-term expression of transgenes in DRG.

The selected polynucleotide sequence is operably linked to control elements that direct the transcription or expression thereof in the subject in vivo. Such control elements can comprise control sequences normally associated with the selected gene.

Typically the vector of the present invention comprises an expression cassette. The term “expression cassette” refers to a nucleic acid construct comprising nucleic acid elements sufficient for the expression of the nucleic acid molecule of the present invention. Typically the nucleic acid molecule encodes a heterologous gene and may also include suitable regulatory elements. The heterologous gene refers to a transgene that encodes an RNA of interest.

One or more expression cassettes may be employed. Each expression cassette may comprise at least a promoter sequence operably linked to a sequence encoding the RNA of interest. Each expression cassette may consist of additional regulatory elements, spacers, introns, UTRs, polyadenylation site, and the like. In some embodiments, the expression cassette is polycistronic with respect to the transgenes encoding e.g. two or more miRNAs. In other embodiments the expression cassette comprises a promoter, a nucleic acid encoding one or more RNA molecules of interest, and a polyA. In further embodiments, the expression cassette comprises 5′-promoter sequence, a sequence encoding a first RNA of interest, a sequence encoding a second RNA of interest, and a polyadenylation sequence-3′.

In some embodiments, an expression cassette may comprise additional elements, for example, an intron, an enhancer, a polyadenylation site, a woodchuck posttranscriptional response element (WPRE), and/or other elements known to affect expression levels of the encoding sequence. Typically, an expression cassette comprises the nucleic acid molecule of the present invention operatively linked to a promoter sequence.

The term “operatively linked” refers to the association of two or more nucleic acid fragments on a single nucleic acid fragment so that the function of one is affected by the other.

For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation.

As used herein, the term “promoter” sequence refers to a polynucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

In some embodiments, the promoter is a heterologous promoter. The term “heterologous promoter”, as used herein, refers to a promoter that is not found to be operatively linked to a given encoding sequence in nature.

Useful heterologous control sequences generally include those derived from sequences encoding mammalian or viral genes. Examples include, but are not limited to, the phophoglycerate kinase (PKG) promoter, CAG (composite of the (CMV) cytomegalovirus enhancer the chicken beta actin promoter (CBA) and the rabbit beta globin intron), U6 promoter, neuronal promoters (Human synapsin 1 (hSyn) promoter, NeuN promoters, CamKII promoter, promoter of Dopamine-1 receptor and Dopamine-2 receptor), the SV40 early promoter, mouse mammary tumor virus LTR promoter; adenovirus major late promoter (Ad MLP); a herpes simplex virus (HSV) promoter, a CMV promoter such as the CMV immediate early promoter region (CMV-IE), rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, CA).

For purposes of the present invention, both heterologous promoters and other control elements, such as DRG-specific and inducible promoters, enhancers and the like, will be of particular use.

An “enhancer” is a polynucleotide sequence that can stimulate promoter activity and may be an innate element of the promoter or a heterologous element inserted to enhance the level or tissue-specificity of a promoter. In some embodiments, the promoter is derived in its entirety from a native gene. In some embodiments, the promoter is composed of different elements derived from different naturally occurring promoters. In some embodiments, the promoter comprises a synthetic polynucleotide sequence. It will be understood by those skilled in the art that different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g. tetracycline-responsive promoters) are well known to those of skill in the art.

In mammalian systems, three kinds of promoters exist and are candidates for construction of the expression vectors: Pol I promoters control transcription of large ribosomal RNAs; Pol II promoters control the transcription of mRNAs (that are translated into protein) and small nuclear RNAs (snRNAs); and Pol III promoters uniquely transcribe small non-coding RNAs. Each has advantages and constraints to consider when designing the construct for expression of the RNAs in vivo. For example, Pol III promoters are useful for synthesizing small interfering RNAs (shRNAs) from DNA templates in vivo. For greater control over tissue specific expression, Pol II promoters are preferred but can only be used for transcription of miRNAs. When a Pol II promoter is used, however, it may be preferred to omit translation initiation signals so that the RNAs function as antisense, siRNA, shRNA or miRNAs and are not translated into peptides in vivo.

The AAV expression vector which harbors the DNA molecule of interest flanked by AAV ITRs, can be constructed by directly inserting the selected sequence (s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publications Nos. WO 92/01070 (published 23 Jan. 1992) and WO 93/03769 (published 4 Mar. 1993). Alternatively, AAV ITRs can be excised from the viral genome or from an AAV vector containing the same and fused 5′ and 3′ of a selected nucleic acid construct that is present in another vector using standard ligation techniques. AAV vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226. Additionally, chimeric genes can be produced synthetically to include AAV ITR sequences arranged 5′ and 3′ of one or more selected nucleic acid sequences. Preferred codons for expression of the chimeric gene sequence in mammalian DRG cells can be used, and in certain embodiments codon optimization of the transgene is performed by well-known methods. The complete chimeric sequence is assembled from overlapping oligonucleotides prepared by standard methods. In order to produce AAV virions, an AAV expression vector is introduced into a suitable host cell using known techniques, such as by transfection. A number of transfection techniques are generally known in the art. Particularly suitable transfection methods include calcium phosphate co-precipitation, direct microinjection into cultured cells, electroporation, liposome mediated gene transfer, lipid-mediated transduction, and nucleic acid delivery using high-velocity microprojectiles.

For instance, a particular viral vector comprises, in addition to a nucleic acid sequence of the invention, the backbone of AAV vector plasmid with ITR derived from AAV-2, the promoter, such as the mouse PGK (phosphoglycerate kinase) gene or the cytomegalovirus/0-actin hybrid promoter (CAG) consisting of the enhancer from the CMV immediate gene, the promoter, splice donor and intron from the chicken 3-actin gene, the splice acceptor from rabbit β-globin, or any neuronal promoter such as the promoter of Dopamine-1 receptor or Dopamine-2 receptor, or the synapsin promoter, with or without the wild-type or mutant form of woodchuck hepatitis virus post-transcriptional regulatory element (WPRE), and a rabbit beta-globin polyA sequence. The viral vector may comprise in addition, a nucleic acid sequence encoding an antibiotic resistance gene such as the genes of resistance ampicillin (AmpR), kanamycin, hygromycin B, geneticin, blasticidin S or puromycin.

In one embodiment, retroviral vectors are employed.

Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types.

In another embodiment, lentiviral vectors are employed.

In a particular embodiment, the invention relates to a lentivirus vector comprising an oligonucleotide sequence that encoding a portion or fragment of FXYD2, or variants thereof.

In another embodiment, the lentivirus vector of the invention comprises any variant of the oligonucleotide sequence which encodes a portion or fragment of FXYD2.

In another embodiment, the lentivirus vector of the invention comprises any variant of the oligonucleotide sequence which encodes for any variant of FXY2D.

In another embodiment, the invention relates to a lentivirus vector comprising an antisense sequence that encodes a portion or fragment of FXYD2, or variants thereof.

In another embodiment, the invention relates to a lentivirus vector comprising a shRNA sequence that encodes a portion or fragment of FXYD2, or variants thereof.

In another embodiment, the invention relates to a lentivirus vector comprising a miRNA sequence that encodes a portion or fragment of FXYD2, or variants thereof.

In another embodiment, the invention relates to a lentivirus vector comprising an antisense oligonucleotide which targets the region comprising or consisting of the nucleotides of SEQ ID NO: 1, SEQ ID NO: 2, SEQ ID NO: 3, SEQ ID NO: 4, SEQ ID NO: 5, or SEQ ID NO: or variants thereof.

In another embodiment, the invention relates to a lentivirus vector comprising one sequence selected from the group consisting of but not limited to SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41 that encodes a portion or fragment of FXYD2, or variants thereof.

In another embodiment, the lentivirus vector of the invention comprises any variant of one sequence selected from the group consisting of but not limited to SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41 which encodes a portion or fragment of FXYD2.

In another embodiment, the lentivirus vector of the invention comprises any variant of one sequence selected from the group consisting of but not limited to SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26.

In another embodiment, the invention relates to a lentivirus vector comprising an oligonucleotide sequence that encodes a portion or fragment of FXYD2, or variants thereof.

In another embodiment, the invention relates to a lentivirus vector comprising a shRNA sequence that encodes a portion or fragment of FXYD2, or variants thereof and a U6 promoter.

In another embodiment, the invention relates to a lentivirus vector comprising an antisense oligonucleotide which targets the region comprising or consisting of the nucleotides of SEQ ID NO: 1, SEQ ID NO: 2, SEQ ID NO: 3, SEQ ID NO: 4, SEQ ID NO: 5, or SEQ ID NO: 6 and a U6 promoter.

In another embodiment, the invention relates to a lentivirus vector comprising the sequence SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41 and a U6 promoter.

In another embodiment, the lentivirus vector of the invention comprises any variant of one sequence selected from the group consisting of but not limited to SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26 and a U6 promoter.

In another embodiment, the invention relates to a lentivirus vector comprising an oligonucleotide sequence that encodes a portion or fragment of FXYD2, or variants thereof and a CAG promoter.

In another embodiment, the invention relates to a lentivirus vector comprising an antisense sequence that encodes a portion or fragment of FXYD2, or variants thereof and a CAG promoter.

In another embodiment, the invention relates to a lentivirus vector comprising a miRNA sequence that encodes a portion or fragment of FXYD2, or variants thereof and a CAG promoter.

In another embodiment, the invention relates to a lentivirus vector comprising a shRNA sequence that encodes a portion or fragment of FXYD2, or variants thereof and a CAG promoter.

In a particular embodiment, the invention relates to a lentivirus vector comprising an antisense oligonucleotide which targets the region comprising or consisting of the nucleotides of SEQ ID NO: 1, SEQ ID NO: 2, SEQ ID NO: 3, SEQ ID NO: 4, SEQ ID NO: 5, or SEQ ID NO: 6 and a CAG promoter.

In a particular embodiment, the invention relates to a lentivirus vector comprising the sequence SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41 and a CAG promoter.

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g.. U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species. Typically, the nucleic acid molecule or the vector of the present invention include “control sequences”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

### Method for Treating Pain

In a third aspect, the invention relates to the inhibitor and/or the antisense oligonucleotide as described above for use in the treatment of pain in a subject in need thereof.

In a particular embodiment, the invention relates to a method for treating a pain in a subject in need thereof comprising a step of administering said subject with a therapeutically effective amount of an inhibitor and/or the antisense oligonucleotide as described above.

In a particular embodiment, the method according to the invention wherein said antisense oligonucleotide targets at least the region comprising or consisting of the nucleotides of SEQ ID NO: 1, SEQ ID NO: 2, or SEQ ID NO: 3 SEQ ID NO: 4, SEQ ID NO: 5 or SEQ ID NO: 6.

In a particular embodiment, the method according to the invention wherein said inhibitor targets a region comprising or consisting of nucleotides of SEQ ID NO: 3.

In a particular embodiment, the method according to the invention wherein said antisense oligonucleotide which comprises or consists of a sequence selected from the group comprising or consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41.

In a particular embodiment, the method according to the invention wherein said antisense oligonucleotide which comprises or consists of a sequence selected from the group comprising or consisting of SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26.

In a particular embodiment, the method according to the invention wherein said antisense oligonucleotide is administered alone (naked) or in a vector as described above.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “pain” refers to an unpleasant feeling often caused by intense or damaging stimuli. The International Association for the Study of Pain's widely used definition states: “Pain is an unpleasant sensory and emotional experience associated with actual or potential tissue damage, or described in terms of such damage”. There are different types of pain: acute pain, chronic pain, somatic pain, nociceptive pain, visceral pain or peripheral pain. In the context of the invention, the pain is peripheral pain. More particularly, the peripheral pain is neuropathic pain, diabetic pain, chemotherapy pain, inflammatory pain, post-surgical pain and/or chronic postoperative pain.

As used herein, the term “subject” denotes a mammal, such as a rodent, a feline, a canine, and a primate. Particularly, the subject according to the invention is a human, a mouse or a rat. As used herein, the term “subject” encompasses “patient”.

In a particular embodiment, the subject suffers or is susceptible to suffer from a pain.

In a particular embodiment, the subject suffers or is susceptible to suffer from peripheral pain.

In a particular embodiment, the subject suffers or is susceptible to suffer from neuropathic pain.

In a particular embodiment, the subject suffers or is susceptible to suffer from inflammatory pain.

In a particular embodiment, the subject suffers or is susceptible to suffer from diabetic pain.

In a particular embodiment, the subject suffers or is susceptible to suffer from chemotherapy pain.

In a particular embodiment, the subject suffers or is susceptible to suffer from post-surgical pain.

In a particular embodiment, the subject suffers or is susceptible to suffer from chronic post-operative pain.

As used herein the terms “administering” or “administration” refer to the act of injecting or otherwise physically delivering a substance as it exists outside the body (e.g., an inhibitor of FXYD2 such as an ASO of the invention) into the subject, such as by, intravenous, intramuscular, enteral, subcutaneous, parenteral, systemic, local, spinal, nasal, topical or epidermal administration (e.g., by injection or infusion). When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof. In a particular embodiment, the administration is performed by a patches, a paste, an ointment, a suspension, a solution or a cream, a gel or a spray. In a particular embodiment, the administration is performed by a cream.

In a particular embodiment, the administration of the inhibitor and/or antisense oligonucleotide is performed by an intrathecal, subcutaneous, topical or intravenous administration.

In a further embodiment, i) an antisense oligonucleotide according to the invention and and a ii) classical treatment for simultaneous, separate or sequential use in the treatment of pain as a combined preparation.

As used herein, the term “classical treatment” refers to any compound, natural or synthetic. In a particular embodiment, the classical treatment is selected from the group consisting of but not limited to: aspirin, paracetamol, Nonsteroidal anti-inflammatory drugs (NSAIDs); codeine, cryotherapy, virtual therapy, cannabis, morphine and its derivatives, opium and its derivatives.

A “therapeutically effective amount” is intended for a minimal amount of active agent (e.g. ASO according to the invention) which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Pharmaceutical Composition

In a fourth aspect, the invention relates to a pharmaceutical composition which comprises the inhibitor and/or the antisense oligonucleotide according to the invention.

In a particular embodiment, the pharmaceutical composition according to the invention wherein said antisense oligonucleotide targets at least the region comprising or consisting of the nucleotides of SEQ ID NO: 1, SEQ ID NO: 2, or SEQ ID NO: 3 SEQ ID NO: 4, SEQ ID NO: 5 or SEQ ID NO: 6.

In a particular embodiment, the pharmaceutical composition according to the invention wherein said inhibitor targets a region comprising or consisting of nucleotides of SEQ ID NO: 3.

In a particular embodiment, the invention relates to the pharmaceutical composition according to the invention comprising at least an antisense oligonucleotide which comprises and/or consists of a sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41.

In a particular embodiment, the the pharmaceutical composition according to the invention wherein said antisense oligonucleotide which comprises or consists of a sequence selected from the group comprising or consisting of SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26.

In a particular embodiment, the invention relates to the pharmaceutical composition according to the invention for use in the treatment of pain.

In a particular embodiment, the invention relates to the pharmaceutical composition for use according to the invention wherein the pain is peripheral pain.

In a particular embodiment, the invention relates to the pharmaceutical composition for use according to the invention wherein the pain is neuropathic pain, diabetic pain, chemotherapy pain, inflammatory pain, surgical pain and/or chronic post-operative pain.

The inhibitor and/or the antisense oligonucleotide as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for per os (oral), sublingual, subcutaneous, intramuscular, intravenous, transdermal, local or rectal administration, the active principle, alone or in combination with another active principle, can be administered in a unit administration form, as a mixture with conventional pharmaceutical supports, to subjects, such as animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

In a particular embodiment, the pharmaceutical composition according to the invention is administered by an intrathecal, subcutaneous, topical or intravenous administration.

Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation may be vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In a particular embodiment, the present invention provides a topical formulation comprising antisense oligonucleotides. For example, and not by way of limitation, the present invention provides a topical formulation comprising antisense oligonucleotides. Dosage forms for the topical or transdermal administration of the inhibitors of the present invention include, but are not limited to, powders, sprays, ointments, pastes, creams, lotions, gels, solutions, patches and inhalants. In certain non-limiting embodiments, a topical formulation comprises antisense oligonucleotides comprised in micelles, liposomes, or non-lipid based microspheres. In certain non-limiting embodiments, such a topical formulation may comprise a permeability enhancing agent such as but not limited to dimethyl sulfoxide, hydrocarbons (for example, alkanes and alkenes), alcohols (for example, glycols and glycerols), acids (for example, fatty acids), amines, amides, esters (for example, isopropyl myristate), surfactants (for example, anionic, cationic, or non-ionic surfactants), terpenes, and lipids (for example, phospholipids).

In a particular embodiment, the formulation is a patches, a paste, an ointment, a suspension, a solution or a cream, a gel or a spray. In a particular embodiment, the formulation is a cream.

The invention will be further illustrated by the following figures and examples. However, these examples and figures should not be interpreted in any way as limiting the scope of the present invention.

### FIGURES

FIG. 1: Identification of antisense oligonucleotides directed against human FXYD2 mRNA. Quantification of FXYD2 protein levels by Western blot of extracts of HEK293 cells after transfection by a series of antisense oligonucleotides directed against human FXYD2 mRNA (A) and by ASO75 and ASO82 with a lengths of 15, 17 and 20 nucleotides each (B). The levels of FXYD2 protein were normalized with actin protein. Data are represented as means±s.e.m. *=p<0.05; **=p<0.01; ***=p<0.001 and ****=p<0.0001, (n=3 replicates).

FIG. 2. In vivo inhibition of Fxyd2 expression in rat DRG neurons by intrathecal injection of lipid-modified Fxyd2 antisense oligonucleotide, Fxyd2-LASO. Increasing amounts of Fxyd2-LASO were intrathecally-injected daily over 14 days (n=3) and lumbar DRGs (L4-L6) tissues were dissected and quantified for Fxyd2 protein levels. The levels of Fxyd2 protein were normalized with actin protein. Data are represented as means±s.e.m. *=p<0.05; n.s., not significant.

FIG. 3. Daily intrathecal injections of lipid-modified Fxyd2 antisense oligonucleotide is at least as effective as intrathecal ω-Conotoxin MVIIA in alleviating pain sensitivity in the SNL model of neuropathic pain. Cohorts of rats were subject to SNL surgery and tested for responses to mechanical stimuli (A: von Frey test; B: Randall-Selitto paw pressure test). Daily intrathecal injections of 2 μg of Fxyd2-LASO (n=9), but not control-LASO (n=9), led to a gradual complete alleviation of mechanical hypersensitivity in the 2 tests, which was reversed by interruption of the treatment. Resumption of Fxyd2-LASO treatment restored the analgesic effect. Treatment of cohort of rats displaying neuropathic pain symptoms with a single intrathecal injection of ω-Conotoxin MVIIA (n=9) alleviated mechanical hypersensitivity for a duration of 1-2 hours. Data are represented as means±s.e.m. *=p<0.05; ***=p<0.001 and ****=p<0.0001.

FIG. 4. Fxyd2-LASO treatment alleviates mechanical hypersensitivity in an inflammatory pain model. Mechanical hypersensitivity was induced by intraplantar injection of CFA (Complete Freund's Adjuvant). Daily injections of Fxyd2-LASO (n=6), but not control-LASO (n=6), alleviated pain symptoms (A: von Frey test; B: Randall-Selitto paw pressure test). The analgesic effect of Fxyd2-LASO was dependent on continued treatment, since interruption of the injections restored the pain hypersensitivity. Data are represented as means±s.e.m. *=p<0.05; **=p<0.01 and ****=p<0.0001.

FIG. 5. Lipid modification of Fxyd2-LASO is essential for its analgesic effect in the SNL model of neuropathic pain. After induction of mechanical hypersensitivity by SNL, rats were intrathecally injected daily with either Fxyd2-LASO (n=6) or Fxyd2-ASO (non-lipid modified form) (n=6). Fxyd2-LASO, but not Fxyd2-ASO, effectively reversed the pain behavior as evidenced by mechanical sensitivity tests (A: von Frey test; B: Randall-Selitto paw pressure test). Data are represented as means±s.e.m. *=p<0.05; **=p<0.01; ***=p<0.001 and ****=p<0.0001.

FIG. 6. Fxyd2 inhibition by intrathecal injection of Accell Fxyd2-siRNA reduces mechanical hypersensitivity in the SNL model. After induction of mechanical hypersensitivity by SNL, animals were intrathecally injected daily with Accell Fxyd2-siRNA (n=9) or Accell control-siRNA (n=9). Accell Fxyd2-siRNA, but not Accell control-siRNA, reduced mechanical hypersensitivity in the von Frey test (A) and the Randall-Selitto paw pressure test (B). Data are represented as means±s.e.m. **=p<0.01; ***=p<0.001 and ****=p<0.0001.

### EXAMPLE

Material & Methods

Animals.

All experiments were approved by the French Ministry of Research (authorization #C34-172-36) and performed according to the guidelines of the International Association for the Study of Pain (IASP). All animals were housed with a 12/12 dark/light cycle with ad libitum access to water and food. Five weekold male Sprague-Dawley rats (Janvier, France), weighing 200 to 250 g at the beginning of the experiments, were used.

Chronic Pain Models.

The SNL (spinal nerve ligation) model of peripheral neuropathic pain and CFA (compete Freund's adjuvant) model of chronic inflammatory pain were used. All surgical procedures were performed under deep isoflurane anesthesia. The SNL procedure was performed as described previously². Briefly, the L6 transverse process was removed to expose the L4 and L5 spinal nerves. The L5 spinal nerve was then isolated and tightly ligated with 6.0 silk thread. The model of complete Freund adjuvant (CFA)-induced pain³ has been used for assessing chronic inflammatory pain. Briefly, under isoflurane anesthesia, an intraplantar injection (50 l) of a solution of 1 mg of Mycobacterium tuberculosis (Sigma-Aldrich) per ml was performed in the left hind-paw of animals.

Intrathecal ASO, LASO, siRNA or ω-Conotoxin MVIIA Injections in Rats.

ASOs and LASOs were manufactured by ChemBioPharm, Bordeaux, France. A non-targeting ASO and LASO were provided by ChemBioPharm (sense sequence: 5′ CGTGTAGGTACGGCAGATC 3′=SEQ ID NO: 32) and used as a negative control. We designed 29 different Fxyd2-ASOs, the sequences are shown in Table 1. An “Accell” siRNA directed against rat-human Fxyd2 mRNA was purchased from Dharmacon. The sense sequence was as follows: 5′ AAGAUUCCGCUGUGGGGGC (UU) 3′ defined by SEQ ID NO: 31. The “Accell Non-targeting in vivo” siRNA (Dharmacon, D-001910-01) was used as a negative control. ω-Conotoxin MVIIA was purchased from Sigma Aldrich (Ref. C1182) and a dose of 100 pmole was intrathecally injected as described by de Souza et al.¹).

SNL-operated rats were tested to confirm mechanical hypersensitivity and were then injected daily intrathecally with 2 μg of control- or Fxyd2-siRNA or ASO or LASO in 20 μl of 5% glucose solution under brief isoflurane anesthesia into the sub-arachnoid space⁴. For CFA-injected rats, 4 μg of control- or Fxyd2-LASO in 20 μl of 5% glucose solution in water were injected daily intrathecally from day 3 after CFA injection.

Behavioral Testing on Rats.

Sprague-Dawley male rats were housed three per cage under standard conditions of light and temperature. Commercial chow pellets and tap water were available ad libitum. After arrival, animals were left to become accustomed to the colony room for 4 days. To avoid stress resulting from the experimental conditions, analyses were performed by the same experimenter in quiet conditions in a test room close to the colony room. For 2 weeks before the experiments, animals were weighed daily, handled gently for 5 min, and placed in the test room for 1 h, where they were left to become accustomed to the nociceptive apparatus. Mechanical allodynia and mechanical hyperalgesia were evaluated days before and the day of surgery (d0) and once daily after surgery. For mechanical allodynia, we performed the von Frey test, as described above for mice, on six animals for each experimental condition. For mechanical hyperalgesia, nociceptive thresholds in handheld rats were determined with the paw-pressure vocalization test as previously described⁵ on six animals for each experimental condition. Briefly, a constantly increasing pressure was applied to the injured hind paw until the rat squeaks. The Basile analgesimeter (Apelex; stylus tip diameter, 1 mm) was used. A 600 g cutoff value was determined to prevent tissue damage.

In Vitro FXYD2 Protein Knockdown Experiments with ASO.

Control-ASO, 29 different Fxyd2-ASOs with lengths of 20 nucleotides each and ASO #75 and ASO #82 with lengths of 15 and 17 nucleotides each were used (the sequences are shown in Table 1) (ChemBioPharm, Bordeaux, France) and tested in vitro in HEK293M cells. HEK293M cells were maintained in DMEM Glutamax (Invitrogen) supplemented with antibiotics (penicillin 50 U/ml, streptomycin 50 μg/ml) and 10% heat-inactivated Fetal Calf Serum. Cells were plated at a density of 50% and treated after 1 day with the indicated ASO for 2 days. Lipofectamine 2000, a cationic lipid (Invitrogen), was used to increase ASO uptake into the cells. Cells were treated with 100 nM of ASO after a preincubation for 20 min with Lipofectamine 2000 diluted at 1/1000 in serum-free OPTI-MEM (Life Technologies). After 4 h, the medium was replaced with standard culture medium described above.

In vivo Fxyd2 protein knockdown experiments with LASO. 0, 0.5, 2, 4 or 8 μg of Fxyd2-LASO in 20 μl of 5% glucose solution were injected daily intrathecally during 14 days. For each concentration of Fxyd2-LASO, 3 rats were injected. Rats received an intraperitoneal injection of pentobarbital and were transcardially perfused with PBS. Lumbar DRGs (L4-L6) were dissected and stored at −80° C.

Western Blot.

Cells or tissues were mechanically homogenized at 4° C. in NP40 buffer (1% NP40, 150 mM NaCl; 50 mM Tris-HCl, pH 7.5 and protease inhibitor). Lysates were clarified for 10 min at 4° C. at 12,000×g. After protein quantification using a BCA kit (Thermofisher, France), lysates were run on SDS-PAGE and transferred to nitrocellulose membrane. Rabbit anti-C-terminal Fxyd2 and mouse anti-actin antibodies were used. After incubation with primary and fluorescent IRDye secondary antibodies (LI-COR Biosciences), immunodetection was performed using Odyssey CLx Imager (LI-COR Biosciences). Quantifications were done with Image Studio Lite software (LI-COR Biosciences).

Statistical Analyses.

For Western blot experiments, statistical analyses were performed using one-way analysis of variance (ANOVA) followed by a post-hoc Dunnett's test. For behavioral studies, group and time effects were validated by two-way ANOVAs for repeated measurements. When ANOVAs showed a significant effect, Bonferroni post-hoc test was used to determine the significance of the differences. P values <0.05 (*), p<0.01 (**), p<0.001 (***) and p<0.0001 (****) were considered as statistically significant. All data presented are means±s.e.m.

Results

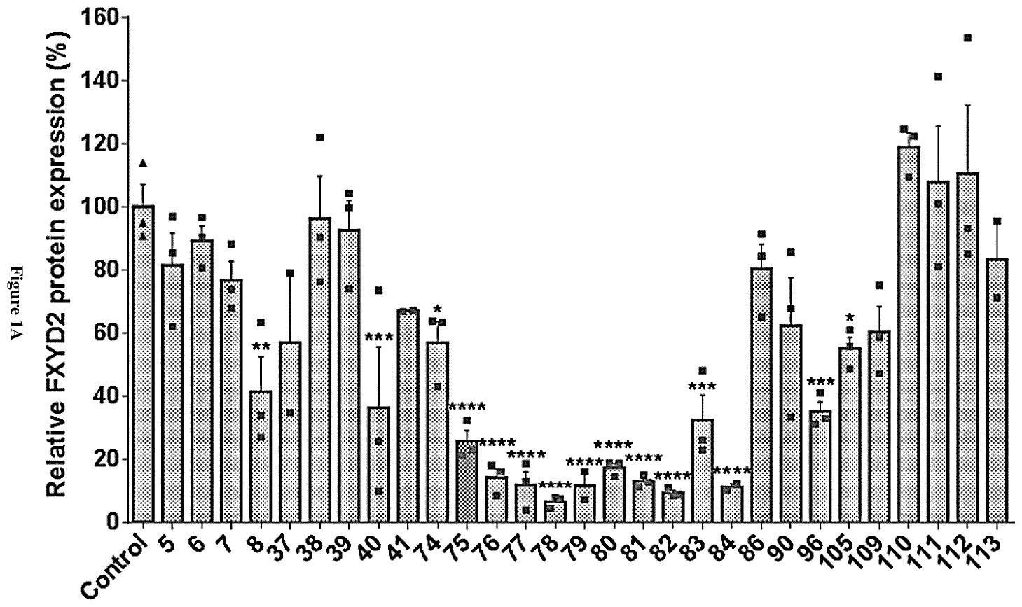

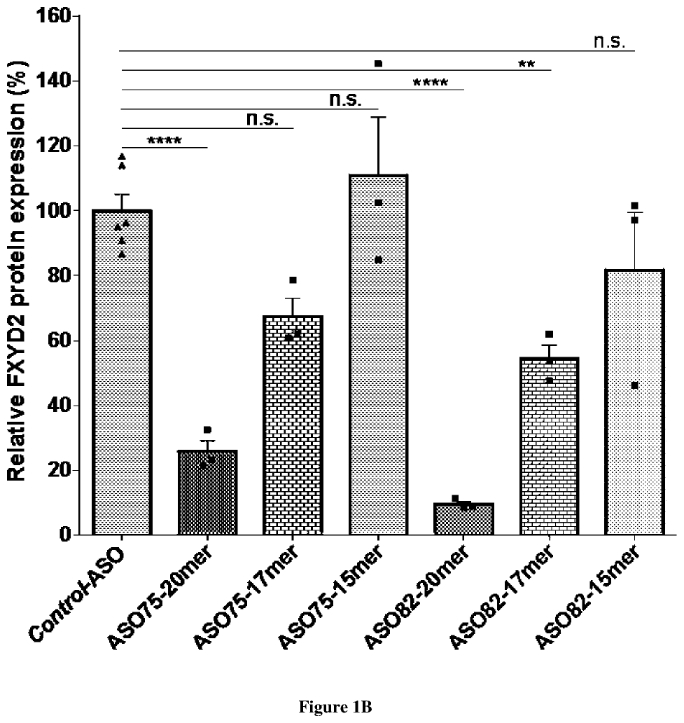

We used the RNAfold program (http://rna.tbi.univie.ac.at/cgi-bin/RNAfold.cgi) to generate a secondary structure of the human FXYD2 mRNA (NCBI Reference sequence NM_001680) that was used to identify oligonucleotide sequences with potential functional knock-down properties (data not shown). A series of overlapping oligonucleotide sequences (Table 1) covering bases 112-267 of the human FXYD2 mRNA was tested by transfecting individual ASOs into human HEK293 cells and quantifying FXYD2 protein levels by Western blot. The region including bases 210-238 (targeted by ASO #75 to #84) was found to be particularly favorable for antisense inhibition (FIG. 1A).

ASO #75 and ASO #82 with lengths of 15, 17 and 20 nucleotides each were tested (SEQ ID NO:17, 24, 38-41). The best inhibition efficiencies were achieved with ASOs with lengths of 20 nucleotides (FIG. 1i).

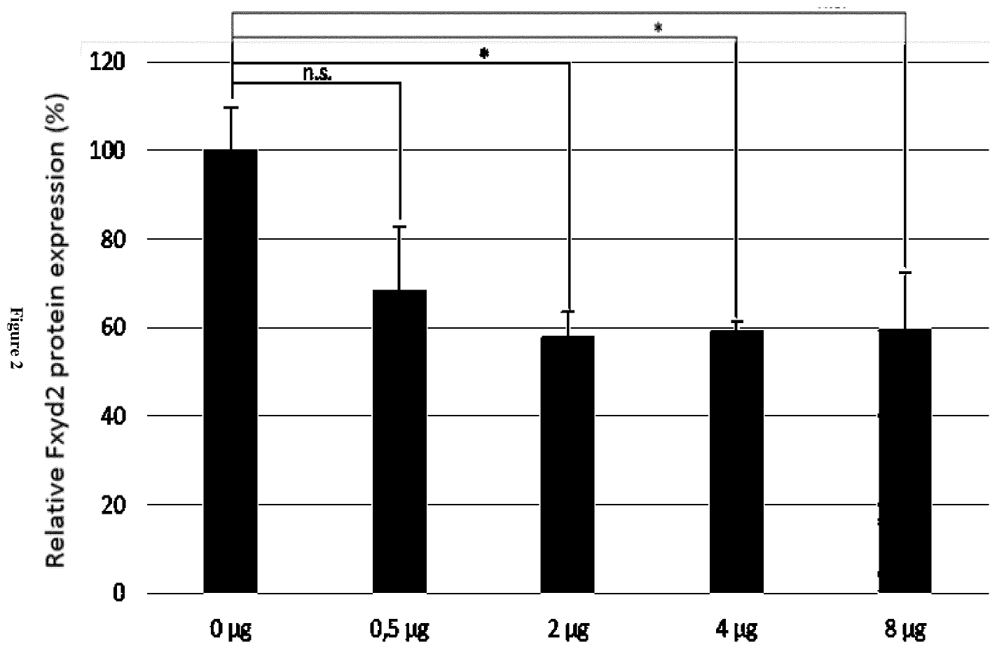

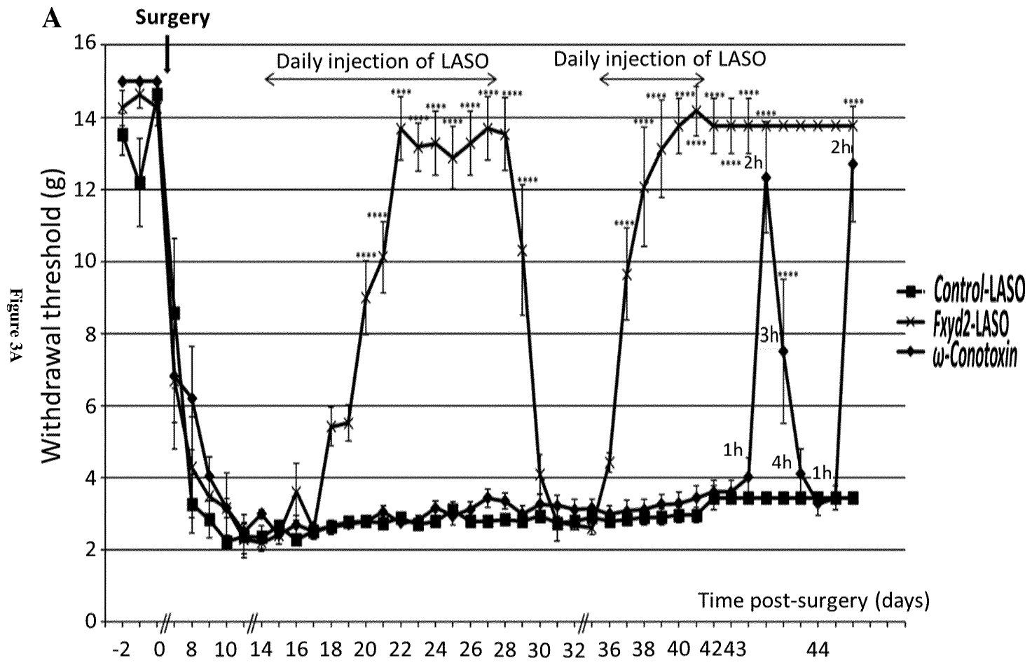

ASO #75 (SEQ ID NO: 17; complementary to bases 210-229) was chosen for further in-depth studies because this sequence is 100% conserved between rat and human mRNAs, thus allowing testing its efficacy in rat model systems. ASO #75 was then tested in vivo for knock-down of the Fxyd2 protein in rat DRG after intrathecal injection (FIG. 2). To avoid known toxic effects of transfection agents and to increase the efficiency of uptake of ASO into neurons, we used a lipid-modified form of ASO #75, hereafter called Fxyd2-LASO. This lipid modification is described in WO2014/195430 and Pokholenko et al 2013.

We first carried out a dose-response analysis to find the minimum amount of Fxyd2-LASO that could effectively decrease Fxyd2 protein levels in DRG neurons. Fxyd2-LASO (0, 0.5, 2, 4 and 8 μg) or control-LASO were intrathecally injected daily for 14 days in 20 μl of 5% glucose solution. Lumbar DRGs (L4, L5, L6) were dissected and the tissue was analyzed by Western blot. FIG. 2 shows that maximal knock-down was achieved by injection of 2 μg of Fxyd2-LASO and no further decrease was observed using higher amounts of LASO. The control-LASO had no effect on Fxyd2 protein levels.

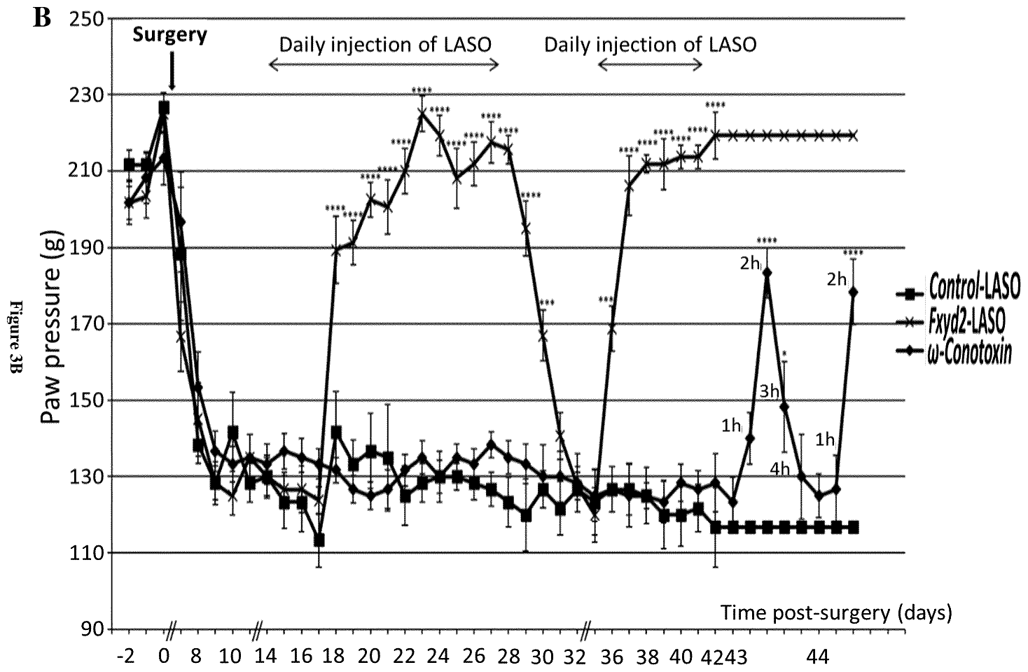

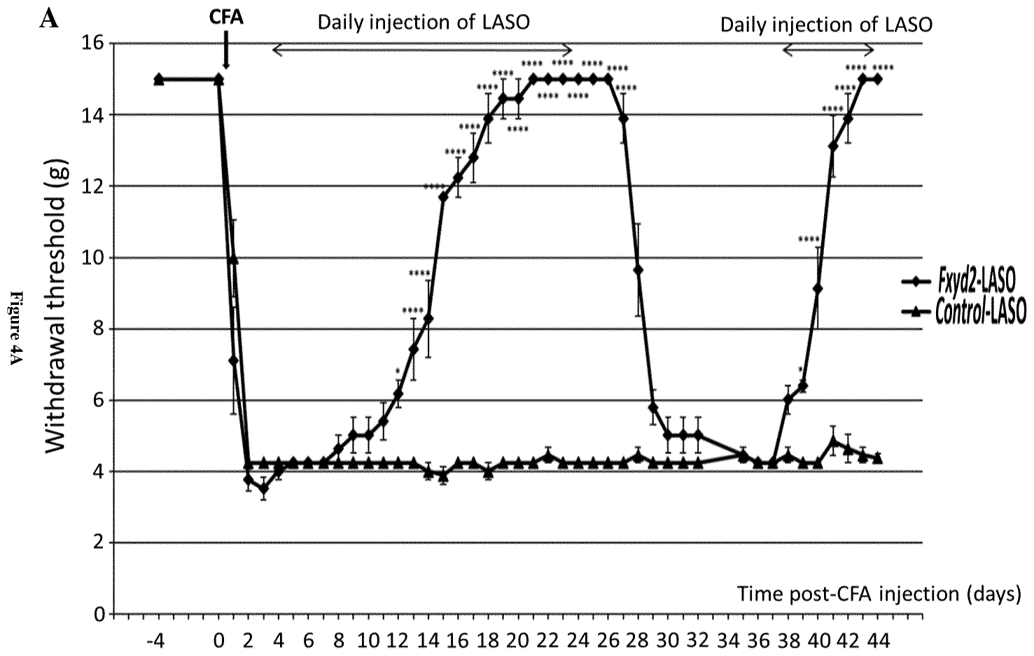

We then tested the in vivo effects of intrathecal injection of Fxyd2-LASO on pain behavior in rats in the SNL model of neuropathic pain (FIG. 3). Four days after surgery rats displayed hypersensitivity to mechanical stimuli as evidenced by lowered withdrawal thresholds to von Frey filaments and increased responses to paw pressure (Randall-Siletto test) on the ipsilateral paw. Daily injections of Fxyd2-LASO from day 14 post-surgery caused a gradual reversal of the pain behavior and the responses had returned to baseline levels by day 21 post-surgery. Complete attenuation of pain behavior was maintained as long as Fxyd2-LASO injections continued (6 days). Interruption of Fxyd2-LASO injections caused a return of hypersensitivity to mechanical stimuli within 2 days. Parallel injections of 2 μg of control-LASO in neuropathic rats had no attenuating effect on pain behavior.

We then compared the analgesic effects of Fxyd2-LASO with the gold standard intrathecally administered medication, ω-Conotoxin MVIIA, of which a synthetic form is commercialized for use in humans under the name Prialt™. ω-Conotoxin MVIIA is a calcium channel inhibitor that is used in certain cases of intractable pain. In the cohort of rats that had been successfully treated with Fxyd2-LASO and in which mechanical hypersensitivity had returned after discontinuation of injection, we commenced a new series of injections. Again, Fxyd2-LASO completely eliminated pain behavior. In parallel, a cohort of rats that had undergone surgery and developed neuropathic pain were treated with ω-Conotoxin MVIIA (100 pmole/intrathecal injection¹). ω-Conotoxin MVIIA partially attenuated the pain behavior (FIG. 3) although, as expected, the duration of the effect was short-lived (1-2 hours). This experiment demonstrates that intrathecal injection of Fxyd2-LASO is an effective analgesic in a rodent model of neuropathic pain, has longer duration of action (days instead of hours) and is at least as effective as the current gold standard, Prialt™.

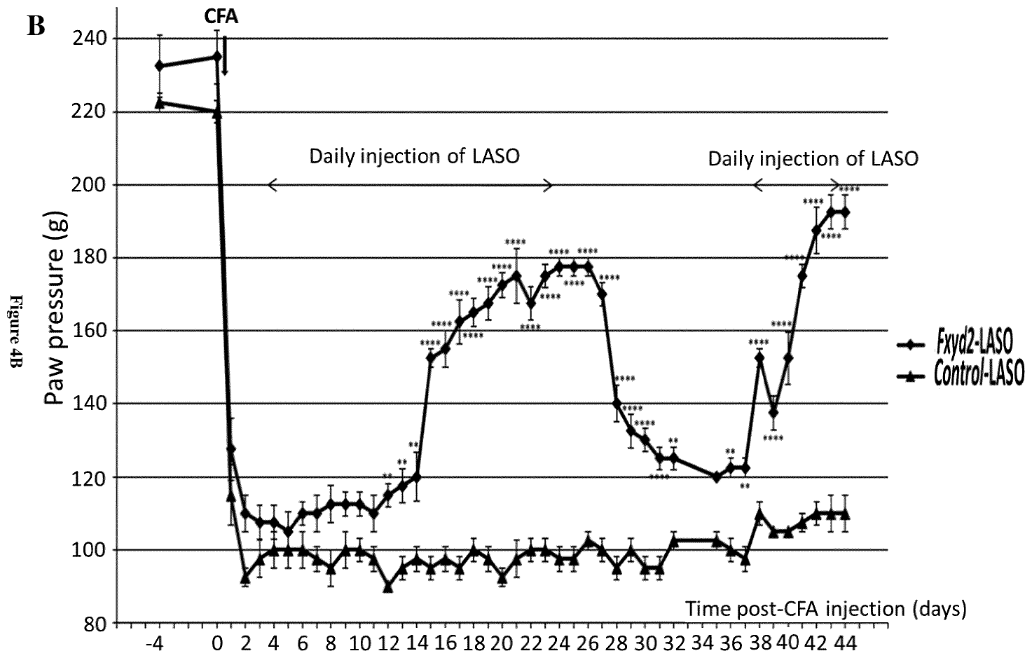

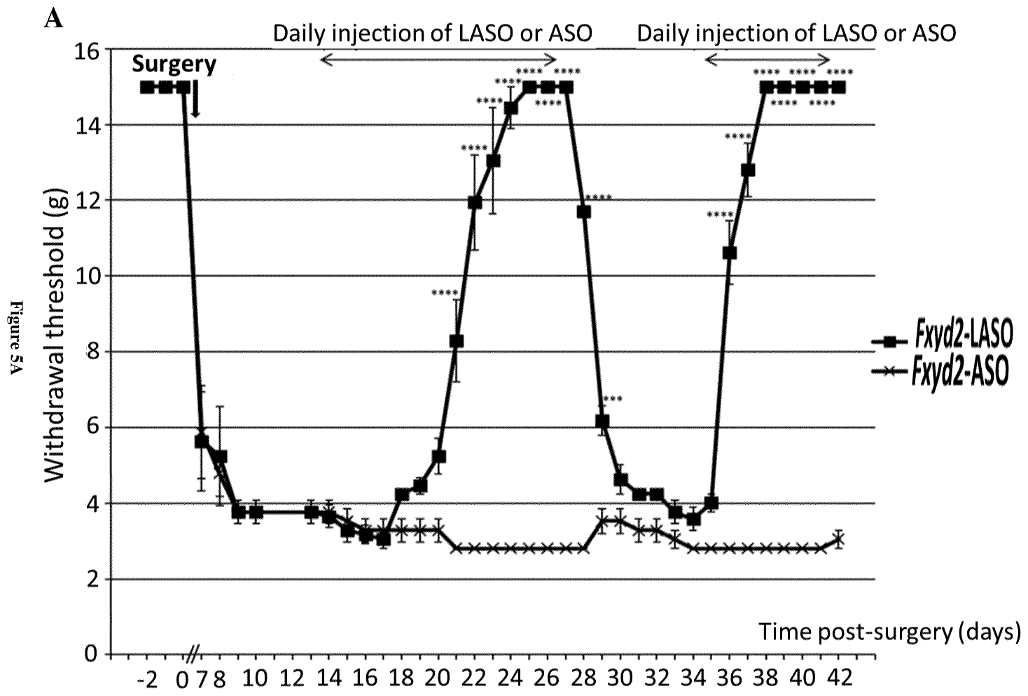

We next tested whether Fxyd2 inhibition by Fxyd2-LASO could be effective in other models of pain, where the underlying mechanisms are known to be different to those pertaining in nerve injury induced pain. We thus employed a commonly-used inflammatory pain model of intraplantar injection of CFA (complete Freund's adjuvant) which causes a long-duration mechanical hypersensitivity (FIG. 4). Again, mechanical sensitivity was tested using the von Frey filaments and the Randall-Selitto test. Injection of CFA caused a rapid mechanical hypersensitivity within 2 days that was maintained over the time-course of the experiment in control animals treated with control-LASO. Single daily intrathecal injection of 4 μg Fxyd2-LASO again attenuated the pain behavior, albeit in a longer time-frame than was the case for nerve injury induced neuropathic pain. The maximum analgesic affect was achieved after 15 days of treatment, compared with 7 days in the injury induced neuropathic pain model. Secondly, although pain behavior was completely attenuated in the von Frey filament test, the results show a partial efficacy in the Randall-Selitto test (FIG. 4). As was the case for the nerve injury model, discontinuation of Fxyd2-LASO injections caused a rapid return of pain behavior and subsequent reinstatement of the Fxyd2-LASO injections restored the analgesic effect.

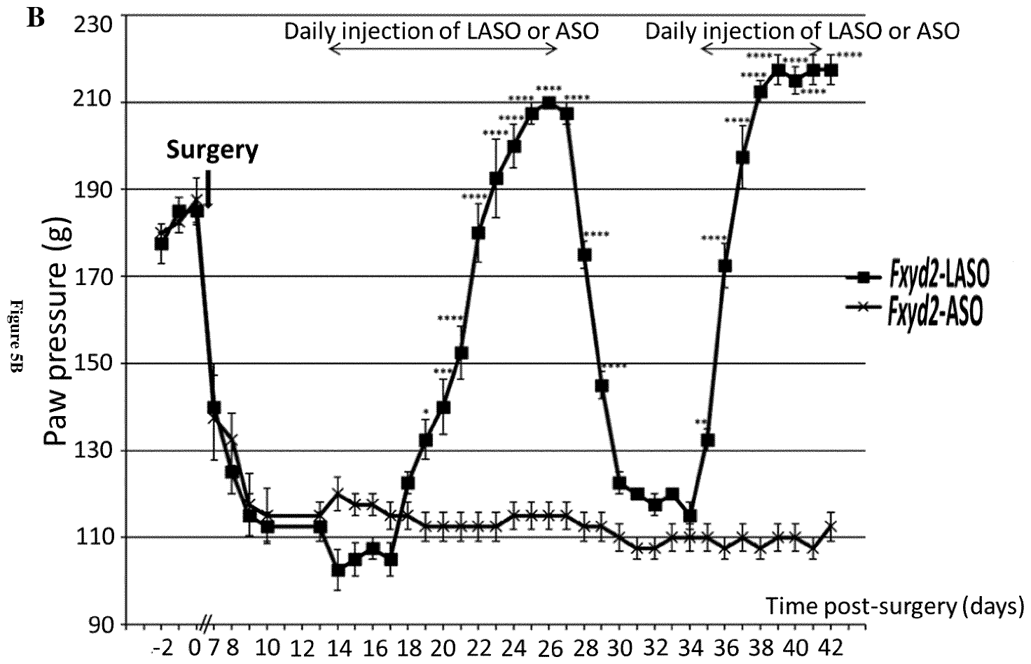

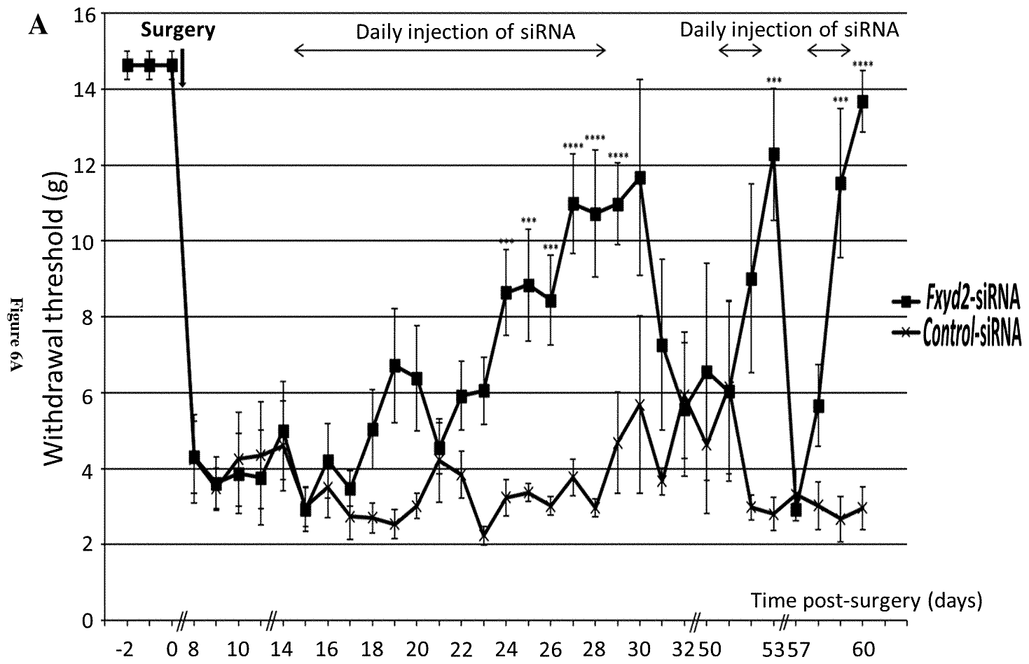

We next explored the importance of the lipid modification of Fxyd2-LASO in its analgesic efficacy, by directly comparing the responses to intrathecal injection of non-modified Fxyd2-ASO antisense oligonucleotide and Fxyd2-LASO in the same experiment (FIG. 5). After SNL surgery and induction of neuropathic pain behavior, cohorts of rats were treated by daily intrathecal injections of 2 μg of Fxyd2-ASO or Fxyd2-LASO. Mechanical sensitivity testing showed that Fxyd2-ASO was inefficient for attenuating pain behaviors, and the lipid modification was necessary for the pain inhibiting effects of the oligonucleotide.

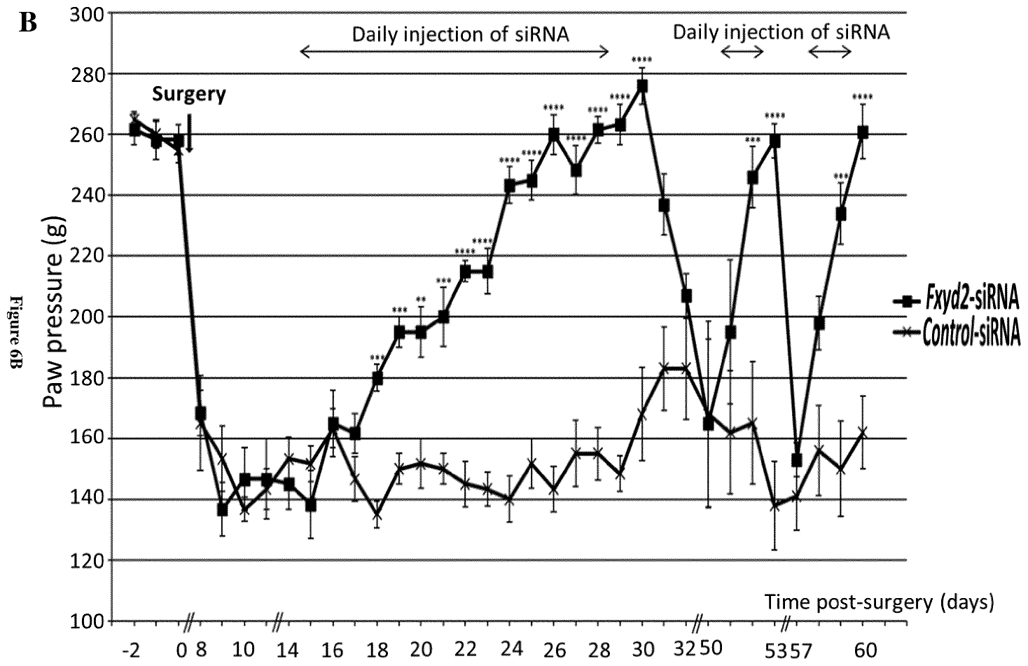

Finally, we tested the efficacy of using a siRNA directed against Fxyd2 mRNA as an alternative method of inhibiting its function (FIG. 6). A custom-made siRNA, synthesized using Accell™ technology (Dharmacon) and directed against the same 100% conserved rat-human sequence as in ASO #75 was purchased. Accell™ technology facilitates cell penetration without added transfectant. We used 2 μg of Fxyd2-siRNA per injection. A “non-targeting” siRNA was used as control. Whilst not as efficient as Fxyd2-LASO, daily Fxyd2-siRNA, but not control siRNA, diminished mechanical hypersensitivity in the SNL model of neuropathic pain.

### CONCLUSION

Overall, these results identify several antisense oligonucleotides that can inhibit human FXYD2 expression in vitro. Intrathecal injection of a lipid-modified form of the 100% conserved rat-human ASO (LASO #75) can dramatically attenuate pain behaviors in two rodent models of neuropathic pain; peripheral nerve injury induced neuropathic pain and CFA induced inflammatory pain.

Accordingly, inventors have demonstrated that antisense oligonucleotides according to the invention are able to inhibit FXYD2 and thus treat pain.

### REFERENCES

Throughout this application, various references describe the state of the art to which this invention pertains. The disclosures of these references are hereby incorporated by reference into the present disclosure.
1—De Souza, A H et al. An evaluation of the antinociceptive effects of Phα1β, a neurotoxin from the spider Phoneutria nigriventer, and ω-conotoxin MVIIA, a cone snail Conus magus toxin, in rat model of inflammatory and neuropathic pain. Cell Mol Neurobiol. 33, 59-67 (2013)
2—Kim, S. H. & Chung, J. M. An experimental model for peripheral neuropathy produced by segmental spinal nerve ligation in the rat. Pain 50, 355-363 (1992).
3—Ferreira, J. et al. Evidence for the participation of kinins in Freund's adjuvant induced inflammatory and nociceptive responses in kinin B1 and B2 receptor knockout mice. Neuropharmacology 41, 1006-1012 (2001).
4—Pieraut, S. et al. NKCC1 phosphorylation stimulates neurite growth of injured adult sensory neurons. J. Neurosci. 25, 6751-6759 (2007).
5—Rivat, C. et al. Non-nociceptive environmental stress induces hyperalgesia, not analgesia, in pain and opioid-experienced rats. Neuropsychopharmacology 32, 2217-2228 (2007).

1. An inhibitor of FXYD2 wherein said inhibitor reduces the expression and/or activity of FXYD2 in a subject in need thereof and targets at least a region comprising or consisting of the nucleotides 219-229 of SEQ ID NO: 3.

2. The inhibitor according to claim 1 wherein said inhibitor targets:
a region comprising or consisting of nucleotides 210-238 of SEQ ID NO:3; and/or
a region comprising or consisting of nucleotides 210-267 of SEQ ID NO:3.

3. The inhibitor according to claim 1, wherein said inhibitor targets at least the region comprising or consisting of the nucleotides of SEQ ID NO: 3.

4. An inhibitor of FXYD2 wherein said inhibitor reduces the expression and/or activity of FXYD2 and targets at least 15 nucleotides of SEQ ID NO: 1, SEQ ID NO: 2, SEQ ID NO: 3, SEQ ID NO: 4, SEQ ID NO: 5 or SEQ ID NO: 6.

5. The inhibitor according to claim 1, wherein the inhibitor is a siRNA, shRNA, an antisense oligonucleotide, miRNA or a ribozyme.

6. The inhibitor according to claim 1, wherein said inhibitor is an antisense oligonucleotide.

7. The inhibitor according to claim 6, wherein said antisense oligonucleotide is lipid-conjugated antisense oligonucleotide (LASO).

8. The inhibitor of claim 6, wherein the antisense oligonucleotide comprises or consists of a sequence set forth as SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40 or SEQ ID NO:41.

9. The inhibitor according to claim 1, wherein the inhibitor reduces the amount of FYXD2 in Dorsal root ganglion (DRG).

10. A method of treating pain in a subject in need thereof, comprising administering to the subject a therapeutically effective amount of the inhibitor of claim 1.

11. The method according to claim 10, wherein the pain is neuropathic pain, diabetic pain, chemotherapy pain, inflammatory pain post-surgical pain and/or chronic postoperative pain.

12. The method of claim 10, wherein the step of administering is performed by intrathecal, subcutaneous, topical, or intravenous administration.

13. A pharmaceutical composition which comprises the inhibitor of claim 1.

14. (canceled)

15. (canceled)

16. The inhibitor according to claim 5, wherein said antisense oligonucleotide comprises modified nucleotides selected from the group consisting of 2′-O-Met, 2′-O-(2-methoxyethyl) (MOE) oligomers and 2′-phosphorothioate analogs.


SEQUENCE LISTING (41 sequences)

SEQ ID NO: 1 - DNA, 593 bp, Homo sapiens
actctccatccaggccccaggcaagcagcacctccctgctctcctgcactcctggacacaaccagcagctcctgccatggacaggtggtacctgggcggcagccccaagggggacgtggacccgttctactatgactatgagaccgttcgcaatgggggcctgatcttcgctggactggccttcatcgtggggctcctcatcctcctcagcagaagattccgctgtgggggcaataagaagcgcaggcaaatcaatgaagatgagccgtaacagcagcctcggcggtgccacccactgcactggggccagctgggaagccaagcatggccctgcctctggcgcctccccttcttccctgggctttagacctttgtccccgtcactgccagcgcttgggctgaaggaagctccagactcaatgtgacccccaggtggcatcgccaactcctgcctcgtgccacctcatgcttataataaagccggcgtcagagaccgctgcttccctcacctgcctgcctgtctccctcctctgtcaccaccagcctctccaagctcaagtacaaatacagccgggtctcatttgttttttcaa

SEQ ID NO: 2 - DNA, 589 bp, Homo sapiens
agacactctccaaaaagcagagacagcaggaagaggggagtggaggcagcccattcacctggggaaatgactgggttgtcgatggacggtggcggcagccccaagggggacgtggacccgttctactatgactatgagaccgttcgcaatgggggcctgatcttcgctggactggccttcatcgtggggctcctcatcctcctcagcagaagattccgctgtgggggcaataagaagcgcaggcaaatcaatgaagatgagccgtaacagcagcctcggcggtgccacccactgcactggggccagctgggaagccaagcatggccctgcctctggcgcctccccttcttccctgggctttagacctttgtccccgtcactgccagcgcttgggctgaaggaagctccagactcaatgtgacccccaggtggcatcgccaactcctgcctcgtgccacctcatgcttataataaagccggcgtcagagaccgctgcttccctcacctgcctgcctgtctccctcctctgtcaccaccagcctctccaagctcaagtacaaatacagccgggtctcatttgttttttcaa

SEQ ID NO: 3 - DNA, 177 bp, Homo sapiens
ggcggcagccccaagggggacgtggacccgttctactatgactatgagaccgttcgcaatgggggcctgatcttcgctggactggccttcatcgtggggctcctcatcctcctcagcagaagattccgctgtgggggcaataagaagcgcaggcaaatcaatgaagatgagccgtaa

SEQ ID NO: 4 - RNA, 593 nt, Homo sapiens
ugagagguagguccgggguccguucgucguggagggacgagaggacgugaggaccuguguuggucgucgaggacgguaccuguccaccauggacccgccgucgggguucccccugcaccugggcaagaugauacugauacucuggcaagcguuacccccggacuagaagcgaccugaccggaaguagcaccccgaggaguaggaggagucgucuucuaaggcgacacccccguuauucuucgcguccguuuaguuacuucuacucggcauugucgucggagccgccacggugggugacgugaccccggucgacccuucgguucguaccgggacggagaccgcggaggggaagaagggacccgaaaucuggaaacaggggcagugacggucgcgaacccgacuuccuucgaggucugaguuacacuggggguccaccguagcgguugaggacggagcacgguggaguacgaauauuauuucggccgcagucucuggcgacgaagggaguggacggacggacagagggaggagacagugguggucggagagguucgaguucauguuuaugucggcccagaguaaacaaaaaaguu

SEQ ID NO: 5 - RNA, 589 nt, Homo sapiens
ucugugagagguuuuucgucucugucguccuucuccccucaccuccgucggguaaguggaccccuuuacugacccaacagcuaccugccaccgccgucgggguucccccugcaccugggcaagaugauacugauacucuggcaagcguuacccccggacuagaagcgaccugaccggaaguagcaccccgaggaguaggaggagucgucuucuaaggcgacacccccguuauucuucgcguccguuuaguuacuucuacucggcauugucgucggagccgccacggugggugacgugaccccggucgacccuucgguucguaccgggacggagaccgcggaggggaagaagggacccgaaaucuggaaacaggggcagugacggucgcgaacccgacuuccuucgaggucugaguuacacuggggguccaccguagcgguugaggacggagcacgguggaguacgaauauuauuucggccgcagucucuggcgacgaagggaguggacggacggacagagggaggagacagugguggucggagagguucgaguucauguuuaugucggcccagaguaaacaaaaaaguu

SEQ ID NO: 6 - RNA, 177 nt, Homo sapiens
ccgccgucgggguucccccugcaccugggcaagaugauacugauacucuggcaagcguuacccccggacuagaagcgaccugaccggaaguagcaccccgaggaguaggaggagucgucuucuaaggcgacacccccguuauucuucgcguccguuuaguuacuucuacucggcauu

SEQ ID NO: 7 - DNA, 20 bp, Homo sapiens
tcatagtagaacgggtccac

SEQ ID NO: 8 - DNA, 20 bp, Homo sapiens
gtcatagtagaacgggtcca

SEQ ID NO: 9 - DNA, 20 bp, Homo sapiens
agtcatagtagaacgggtcc

SEQ ID NO: 10 - DNA, 20 bp, Homo sapiens
tagtcatagtagaacgggtc

SEQ ID NO: 11 - DNA, 20 bp, Homo sapiens
agtccagcgaagatcaggcc

SEQ ID NO: 12 - DNA, 20 bp, Homo sapiens
cagtccagcgaagatcaggc

SEQ ID NO: 13 - DNA, 20 bp, Homo sapiens
ccagtccagcgaagatcagg

SEQ ID NO: 14 - DNA, 20 bp, Homo sapiens
gccagtccagcgaagatcag

SEQ ID NO: 15 - DNA, 20 bp, Homo sapiens
ggccagtccagcgaagatca

SEQ ID NO: 16 - DNA, 20 bp, Homo sapiens
cccccacagcggaatcttct

SEQ ID NO: 17 - DNA, 20 bp, Homo sapiens
tgcccccacagcggaatctt

SEQ ID NO: 18 - DNA, 20 bp, Homo sapiens
ttgcccccacagcggaatct

SEQ ID NO: 19 - DNA, 20 bp, Homo sapiens
attgcccccacagcggaatc

SEQ ID NO: 20 - DNA, 20 bp, Homo sapiens
tattgcccccacagcggaat

SEQ ID NO: 21 - DNA, 20 bp, Homo sapiens
ttattgcccccacagcggaa

SEQ ID NO: 22 - DNA, 20 bp, Homo sapiens
cttattgcccccacagcgga

SEQ ID NO: 23 - DNA, 20 bp, Homo sapiens
tcttattgcccccacagcgg

SEQ ID NO: 24 - DNA, 20 bp, Homo sapiens
ttcttattgcccccacagcg

SEQ ID NO: 25 - DNA, 20 bp, Homo sapiens
cttcttattgcccccacagc

SEQ ID NO: 26 - DNA, 20 bp, Homo sapiens
gcttcttattgcccccacag

SEQ ID NO: 27 - DNA, 20 bp, Homo sapiens
cggctcatcttcattgattt

SEQ ID NO: 28 - DNA, 20 bp, Homo sapiens
acggctcatcttcattgatt

SEQ ID NO: 29 - DNA, 20 bp, Homo sapiens
tacggctcatcttcattgat

SEQ ID NO: 30 - DNA, 20 bp, Homo sapiens
ttacggctcatcttcattga

SEQ ID NO: 31 - RNA, 21 nt, Homo sapiens
aagauuccgcugugggggcuu

SEQ ID NO: 32 - DNA, 19 bp, Homo sapiens
cgtgtaggtacggcagatc

SEQ ID NO: 33 - DNA, 20 bp, Homo sapiens
gcgcttcttattgcccccac

SEQ ID NO: 34 - DNA, 20 bp, Homo sapiens
gcctgcgcttcttattgccc

SEQ ID NO: 35 - DNA, 20 bp, Homo sapiens
tgatttgcctgcgcttctta

SEQ ID NO: 36 - DNA, 20 bp, Homo sapiens
catcttcattgatttgcctg

SEQ ID NO: 37 - DNA, 20 bp, Homo sapiens
ggctcatcttcattgatttg

SEQ ID NO: 38 - DNA, 15 bp, Homo sapiens
ccacagcggaatctt

SEQ ID NO: 39 - DNA, 17 bp, Homo sapiens
ccccacagcggaatctt

SEQ ID NO: 40 - DNA, 15 bp, Homo sapiens
tcttattgcccccac

SEQ ID NO: 41 - DNA, 17 bp, Homo sapiens
tcttattgcccccacag